(19) United States
(12) Patent Application Publication
(10) Pub. No.: US 2023/0311072 A1
(43) Pub. Date: Oct. 5, 2023

(54) SELECTION OF CROSSLINKERS AND CONTROL OF MICROSTRUCTURE OF VAPOUR-PHASE CROSSLINKED COMPOSITE MEMBRANES FOR ORGANIC SOLVENT SEPARATION

(71) Applicant: National University of Singapore, Singapore (SG)

(72) Inventors: Zhuofan Gao, Singapore (SG); Tai-Shung Chung, Singapore (SG); Ali Naderi, Singapore (SG); Wan Wei, Singapore (SG)

(73) Assignee: National University of Singapore, Singapore (SG)

(21) Appl. No.: 18/006,293

(22) PCT Filed: Jul. 19, 2021
(86) PCT No.: PCT/SG2021/050423

§ 371 Date: Jan. 20, 2023

(30) Foreign Application Priority Data
Jul. 21, 2020 (SG) 10202006944R

(51) Int. Cl. B01D 71/64; B01D 67/00; B01D 69/14; B01D 61/02; B01D 69/02; B01D 71/02; C07C 29/76; B01D 69/08

(52) U.S. Cl. CPC B01D 69/148; B01D 61/025; B01D 61/027; B01D 67/00793; B01D 69/02; B01D 69/087; B01D 69/1411; B01D 71/028; B01D 71/64; C07C 29/76; B01D 2323/30; B01D 2325/02831; B01D 2325/02832; B01D 2325/04; B01D 2325/30; B01D 2325/34

(57) ABSTRACT

Disclosed herein are vapour-phase crosslin ked composite membranes in the form of crosslinked polymers and defined inorganic materials. The membranes disclosed herein may have a narrow pore size distribution and precise molecule separation ability and may be used for organic solvent nanofiltration and organic solvent reverse osmosis. Also disclosed herein are methods of forming the membranes, and filtration. In a preferred embodiment, the vapour-phase crosslinked composite membrane is obtained by exposing a composite membrane comprising polyimide and UiO-66-NH₂ particles to an amine vapour.

### FIELD OF INVENTION

The current invention relates to the field of crosslinked composite membranes. These membranes contain a polymeric matrix and an inorganic material. These membranes may be useful for organic solvent nanofiltration and organic solvent reverse osmosis.

### BACKGROUND

The listing or discussion of a prior-published document in this specification should not necessarily be taken as an acknowledgment that the document is part of the state of the art or is common general knowledge.

To comply with the strict requirements of sustainability and to achieve cost-effective industrial processes, polymer-based organic solvent nanofiltration (OSN) membranes have received significant attention recently for separations involving organic solvents. The OSN process aims to fractionate small molecules with a molecular weight (MW) of 200 - 1000 g mol⁻¹ from organic solvents (P. Marchetti et al., Chem. Rev. 2014, 114, 10735-10806; P. Vandezande, L. E. Gevers & I. F. J. Vankelecom, J. Chem. Soc. Rev. 2008, 37, 365-405; X. Q. Cheng et al., Adv. Polym. Technol. 2014, 33, 21455; and M. Galizia & K. P. Bye, Front. Chem. 2018, 6, 511). Polymeric materials with outstanding chemical resistance either intrinsically or gained via chemical or thermal modifications have been explored in a wide range of solvents and developed into membranes.

A narrow pore size distribution is essential for membranes to have a sharp molecular weight cut-off (MWCO) and high rejections towards targeted solutes with a specific molecular weight, molecular size or functional group. However, current solvent-resistant membranes often lack a narrow pore size distribution and the ability for precise molecule separation. In addition, permeance and selectivity are two critical parameters to assess the membrane performance, and a trade-off relation between permeance (flux) and selectivity (rejection) is often observed.

Therefore, there is a need to develop new sustainable, greener and scalable OSN membranes that have a sharp MWCO and high selectivity as both the biochemical and pharmaceutical industries have high demands for OSN membranes with such characteristics. Similar properties are also required by organic solvent reverse osmosis (OSRO) membranes, for which there is also a need.

### SUMMARY OF INVENTION

It has been surprisingly found that some or all of the above-mentioned problems can be solved using membranes that have been crosslinked using polyamines. Aspects and embodiments of the invention will now be discussed with reference to the following numbered embodiments.

1. An organic solvent nanofiltration (OSN) membrane and/or an organic solvent reverse osmosis (OSRO) membrane comprising a mixed matrix material that comprises:
a solvent-resistant polymeric matrix with functional groups suitable for crosslinking; and
an inorganic material homogeneously dispersed throughout the polymeric matrix, where the mixed matrix material has a first region comprising a surface of the membrane and a second region, wherein:
   the first region of the mixed matrix material further comprises a crosslinking agent that crosslinks the polymeric matrix in said first region via the functional groups suitable for crosslinking; and
   the first region has a mean effective pore size of from 0.2 to 2 nm.

2. The membrane according to Clause 1, wherein the solvent-resistant polymeric matrix with functional groups suitable for crosslinking is a polymeric matrix formed from one or more of the group selected from a polybenzimidazole (PBI), a poly(ether ether ketone) (PEEK), a polyacrylonitrile (PAN), and a polyimide (PI), optionally wherein the solvent-resistant polymeric matrix with functional groups suitable for crosslinking is a polymeric matrix formed from one or more of the group selected from a polybenzimidazole (PBI) and a polyimide (PI).

3. The membrane according to Clause 2, wherein the polymeric matrix is formed by a polyimide.

4. The membrane according to Clause 3, wherein the polyimide has a repeating unit of formula I:

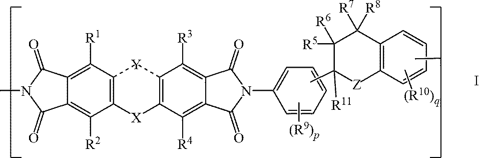

each of R¹ to R⁸ and R¹¹ is independently selected from H, C₁₋₃ alkyl, C₁₋₃ haloalkyl and halo;
X is a bond or is —C(═O)—;
Y is absent or is —C(═O)—; or
X and Y are each absent so as to form a fused aromatic structure of formula (Ia):

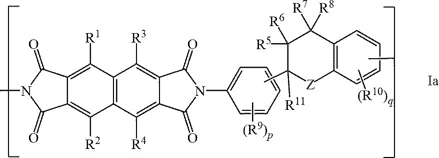

each R⁹, when present, is independently selected from the group comprising: C₁₋₃ alkyl, C₁₋₃ haloalkyl and halo;
each R¹⁰, when present, is independently selected from the group comprising: C₁₋₃ alkyl, C₁₋₃ haloalkyl and halo;
Z is selected from the group comprising: a bond and —C(R¹)₂—;
p is from 0 to 4; and
q is from 0 to 3.

5. The membrane according to Clause 4, wherein the polyimide has a repeating unit of formula II:

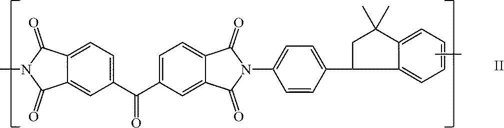

6. The membrane according to any one of the preceding clauses, wherein the inorganic material is selected from one or more of a metal organic framework (MOF), carbon nanotubes, zeolites, titanium dioxide (TiO₂), nanoalumina, silica nanoparticles, silver nanoparticles, and a graphene oxide.

7. The membrane according to Clause 6, wherein the inorganic material is a MOF.

8. The membrane according to Clause 7, wherein the MOF has one or more of the following properties:
(a) a porosity of greater than or equal to 50% of the MOF crystal volume, such as from 55 to 80%, such as from 60 to 70%;
(b) a surface area of from 1,000 to 10,000 m²/g, such as from 2,000 to 8,000 m²/g, such as from 4,000 to 6,000 m²/g;
(c) a diameter of the particle of from 10 to 1,000 nm, such as from 25 to 500 nm, such as from 50 to 150 nm.

9. The membrane according to Clause 7 or Clause 8, wherein the MOF is selected from one or more of a UiO-66 MOF (e.g. UiO-66-COOH or, more particularly, UiO-66, UiO-66-COOH, UiO-66-F₄ and UiO-66-NH₂), a MIL-53(AI) MOF, a ZIF-8 MOF, and a HKUST-1 MOF.

10. The membrane according to any one of the preceding clauses, wherein the weight to weight ratio of the polyimide polymeric matrix material to the inorganic material is from 100:1 to 1000:1, such as from 100:1 to 25:1, such as from 150:1 to 200:1, such as 180:1.

11. The membrane according to any one of the preceding clauses, wherein the polymeric matrix material is partially crosslinked by a diamine (e.g. 1,6-hexanediamine), optionally wherein the polymeric matrix material is a polyimide polymeric matrix material is partially crosslinked by a diamine (e.g. 1,6-hexanediamine).

12. The membrane according to any one of the preceding clauses, wherein the crosslinking agent is selected from one or more of the group consisting of a polyamine, and a hydrazine.

13. The membrane according to Clause 12, wherein crosslinking agent is a polyamine.

14. The membrane according to Clause 11 or Clause 12, wherein the polyamine is selected from one or more of the group consisting of ethylenediamine (EDA), trimethylamine (TEA), or more particularly, tris(2-aminoethyl)amine (TAEA), N,N′-bis(3-aminopropyl)-1,3-propanediamine (APPD), 1,4-bis(3-aminopropyl) piperazine (BAPP), and polyethylenimine (PEI), optionally wherein:

the polyamine is selected from one or more of the group consisting of N,N′-bis(3-aminopropyl)-1,3-propanediamine (APPD), 1,4-bis(3-aminopropyl) piperazine (BAPP), and polyethylenimine (PEI).

15. The membrane according to Clause 14, wherein the polyamine is selected from APPD or PEI, optionally wherein, when present, PEI has a weight average molecular weight of from 100 to 1,000 g mol⁻¹, such as 800 g mol⁻¹.

16. The membrane according to any one of the preceding clauses, wherein the first region has a mean effective pore size of from 0.4 to 1.2 nm, such as from 0.5 to 1.13 nm, such as 0.6 to 0.9 nm.

17. The membrane according to any one of the preceding clauses, wherein the mixed matrix material has a thickness of from 20 to 150 µm, such as from 25 to 110 µm.

18. The membrane according to any one of the preceding clauses, wherein the first region has a thickness of from 100 to 5,000 nm, such as from 200 to 3,500 nm, such as from 300 to 3,000 nm, such as from 500 to 2,500 nm.

19. The membrane according to any one of the preceding clauses, wherein the membrane further comprises a non-woven substrate.

20. The membrane according to Clause 19, wherein the non-woven substrate is formed from one or more from the group consisting of a polyester (PET) and a polypropylene (PP).

21. The membrane according to Clause 20, wherein the non-woven substrate is formed from polyethylene terephthalate.

22. The membrane according to any one of the preceding clauses, wherein the membrane displays a molecular weight cut-off value of from 100 to 500 Daltons, optionally wherein:
(a) when the membrane is an OSN membrane it displays a molecular weight cut-off value of from 400 to 500 Daltons or when the membrane is an OSRO membrane it displays a molecular weight cut-off value of less than or equal to 200 Daltons, such as from 100 to 200 Daltons; and/or
(b) a rejection rate for a material having a molecular weight that is at least 10% above the molecular weight cut-off value of the membrane is at least 90%.

23. The membrane according to any one of the preceding clauses, wherein the membrane is suitable for use in nanofiltration applications using one or more solvents selected from two or more of the solvent classes: polar protic, polar aprotic and non-polar organic solvents.

24. The membrane according to Clause 23, wherein the membrane is suitable for use in nanofiltration applications using one or more solvents selected from polar protic, polar aprotic and non-polar organic solvents.

25. The membrane according to any one of the preceding clauses, wherein the membrane has a pure solvent permeance value of from 0.1 to 4 L m⁻² h⁻¹ bar⁻¹, optionally wherein, the membrane has one or more of the following pure solvent permeance values:
(a) from 2 - 4 L m⁻² h⁻¹ bar⁻¹ for methanol;
(b) from 0.5 - 1.5 L m⁻² h⁻¹ bar⁻¹ for isopropanol (IPA);
(c) from 0.6 - 4 L m⁻² h⁻¹ bar⁻¹ for hexane;
(d) from 0.9 - 1.5 L m⁻² h⁻¹ bar⁻¹ for toluene;
(e) from 0.8-1.3 L m⁻² h⁻¹ bar⁻¹ for tetrahydrofuran (THF);
(f) from 0.2-1 L m⁻² h⁻¹ bar⁻¹ for dimethylformamide (DMF); and
(g) from 1 - 2 L m⁻² h⁻¹ bar⁻¹ for ethanol.

26. A method of filtration using an organic solvent nanofiltration (OSN) membrane as described in any one of Clauses 1 to 25, comprising the steps of
(a) providing a solution comprising a first compound having a first molecular weight and a second compound having a second molecular weight; and
(b) subjecting the solution to filtration using an OSN membrane as described in any one of Clauses 1 to 25, such that the first and second compounds are separated from one another, wherein
   the first molecular weight is lower than the second molecular weight and the OSN membrane has a molecular weight cut-off that prevents the second compound from passing through the membrane, thereby separating the first and second compounds.

27. The method according to Clause 26, wherein the first and second compounds are solvents.

28. The method according to Clause 26, wherein the method further comprises an organic solvent and the first and second compounds are not organic solvents.

29. A method of forming an organic solvent nanofiltration (OSN) membrane and/or an organic solvent reverse osmosis (OSRO) membrane comprising a mixed matrix material as described in any one of Clauses 1 to 25, wherein the method comprises the steps of:
(a) providing a material comprising:
   a partially crosslinked solvent-resistant polymeric matrix with functional groups suitable for crosslinking; and
   an inorganic material homogeneously dispersed throughout the polymeric matrix, where the material has a first region comprising a surface of the material and a second region; and
(b) exposing the first region of the material to a vapour comprising a crosslinking agent that generates further crosslinks in said first region to provide the organic solvent nanofiltration (OSN) membrane.

30. A method of filtration using an organic solvent reverse osmosis (OSRO) membrane as described in any one of Clauses 1 to 25, comprising the steps of
(a) providing a solution comprising a first solvent having a first molecular weight and a second solvent having a second molecular weight; and
(b) subjecting the solution to filtration using an OSRO membrane as described in any one of Clauses 1 to 25, such that the first and second solvents are separated from one another, wherein
   the first molecular weight is lower than the second molecular weight and the OSRO membrane has a molecular weight cut-off that prevents the second solvent from passing through the membrane, thereby separating the first and second solvents.

31. A method of forming an organic solvent reverse osmosis (OSRO) membrane comprising a mixed matrix material as described in any one of Clauses 1 to 25, wherein the method comprises the steps of:
(a) providing a material comprising:
   a partially crosslinked solvent-resistant polymeric matrix with functional groups suitable for crosslinking; and
   an inorganic material homogeneously dispersed throughout the polymeric matrix, where the material has a first region comprising a surface of the material and a second region; and
(b) exposing the first region of the material to a vapour comprising a crosslinking agent that generates further crosslinks in said first region to provide the organic solvent reverse osmosis (OSRO) membrane.

### DRAWINGS

FIG. 1 shows the 3D and chemical structures of (1) N,N′-Bis(3-aminopropyl)-1,3-propanediamine (APPD); (2) 1,4-Bis(3-aminopropyl)piperazine (BAPP); and (3) Polyethylenimine (PEI), simulated by Materials Studio 2018 version.

FIG. 2 depicts the OSN performance testing setup.

FIG. 3 depicts the pure isopropanol (IPA) permeance and Rose Bengal (RB) rejection in IPA of (a) APPD-; (b) BAPP-; and (c) PEI-modified vapour-phase crosslinking (VPC)/mixed matrix membranes (MMMs) as a function of reaction duration (Feed: pure IPA and 50 ppm RB in IPA; Pressure: 10.0 bar).

FIG. 4 shows the surface and cross-sectional field emission scanning electron microscopy (FESEM) images of BAPP-modified MMMs with 0.1 wt% UiO-66-NH₂ as a function of reaction duration: 10 min, 20 min, 30 min and 45 min (Reaction temperature: 120° C.; Pressure: 1 atm).

FIG. 5 depicts the pore size distributions of the BAPP-modified MMMs as a function of reaction duration deduced by the probability density function (µₚ: the mean effective pore size).

FIG. 6 depicts the R parameter of the crosslinked MMM substrate (S1) and BAPP-modified MMMs as a function of VPC reaction duration and positron incident energy.

FIG. 7 depicts the proposed possible chemical reacting mechanisms between the Matrimid® polymer and amine crosslinking monomers.

FIG. 8 depicts the FTIR spectra of pristine crosslinked MMM substrate and BAPP-modified MMMs as a function of crosslinking duration.

FIG. 9 shows the surface and cross-sectional FESEM images of APPD-modified MMMs with 0.1 wt% UiO-66-NH₂ as a function of reaction duration: 10 min, 20 min, 30 min and 45 min (Reaction temperature: 120° C.; Pressure: 1 atm).

FIG. 10 depicts the surface and cross-sectional FESEM images of PEI-modified MMMs with 0.1 wt% UiO-66-NH₂ as a function of reaction duration: 10 min, 20 min, 30 min and 45 min (Reaction temperature: 140° C.; Pressure: 0.1 bar).

FIG. 11 depicts the pore size distributions of the 30-min VPC/MMMs deduced by probability density function (µₚ: the mean effective pore size).

FIG. 12 shows the FESEM images of MMM/PET substrates: (a) cast on glass plates with a gap of 150 µm, then IPA bath; (b) cast on PET fabrics with a gap of 150 µm, then IPA bath; (c) cast on PET fabrics with a gap of 150 µm, then water bath; and (d) cast on PET fabrics with a gap of 100 µm, then water bath.

FIG. 13 depicts the FTIR spectra of various MMM substrates: (a) cast on glass plates with a gap of 150 µm, then IPA bath; (b) cast on PET fabrics with a gap of 150 µm, then IPA bath; (c) cast on PET fabrics with a gap of 150 µm, then water bath; and (d) cast on PET fabrics with a gap of 100 µm, then water bath.

FIG. 14 shows the FESEM images of the unmodified MMM/PET and VPC modified MMM/PET membranes for 20 min of VPC duration.

FIG. 15 depicts the dye rejection curves of VPC/MMM/PET hybrid membranes modified by different amine vapour for 20 min and tested in IPA at 10 bar and room temperature.

FIG. 16 depicts the 3D molecular structures, dimensions and molecular volumes of neutral solutes.

FIG. 17 depicts the pure solvent permeances tested at 10 bar and room temperature as a function of (a) solvent species; and (b) solvent properties in terms of MV·η⁻¹ for VPC/MMM/PET membranes modified by different amine vapour for 20 min.

FIG. 18 depicts the permeance (solid dot) and TC rejection (hollow dot) of (a) APPD-modified membrane (S4-N-APPD-20); (b) BAPP-modified membrane (S4-N-BAPP-20); and (c) PEI-modified membrane (S4-N-PEI-20) in 120-hour OSN tests using 50 ppm TC in IPA, EtOH, THF and DMF as feeds at 5 bar and room temperature.

FIG. 19 depicts the schematic diagram of the hollow fiber spinning spinneret.

FIG. 20 depicts the experimental setup of vapor phase modification (HFs: hollow fibers).

FIG. 21 depicts (a) schematic; and (b) photograph of the lab-scale crossflow OSN hollow fiber setup.

### DESCRIPTION

In a first aspect of the invention, there is provided an organic solvent nanofiltration (OSN) membrane and/or an organic solvent reverse osmosis (OSRO) membrane (e.g. an organic solvent nanofiltration (OSN) membrane) comprising a mixed matrix material that comprises:
a solvent-resistant polymeric matrix with functional groups suitable for crosslinking; and
an inorganic material homogeneously dispersed throughout the polymeric matrix, where the mixed matrix material has a first region comprising a surface of the membrane and a second region, wherein:
   the first region of the mixed matrix material further comprises a crosslinking agent that crosslinks the polymeric matrix in said first region via the functional groups suitable for crosslinking; and
   the first region has a mean effective pore size of from 0.2 to 2 nm.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

“Solvent-resistant” when used herein refers to the ability of a polymeric matrix to resist dissolution into a solvent (e.g. an organic solvent) it is contacted with. In certain embodiments, the solvent resistance may be generated by the presence of crosslinks throughout the polymeric matrix.

Any suitable solvent-resistant polymeric matrix with functional groups suitable for crosslinking may be used herein. For example, the solvent-resistant polymeric matrix with functional groups suitable for crosslinking may be a polymeric matrix formed from one or more of the group selected from a polybenzimidazole (PBI), a poly(ether ether ketone) (PEEK), a polyacrylonitrile (PAN), and a polyimide (PI), optionally wherein the solvent-resistant polymeric matrix with functional groups suitable for crosslinking is a polymeric matrix formed from one or more of the group selected from a polybenzimidazole (PBI) and a polyimide (PI). More particularly, the polymeric matrix may be formed by a polyimide.

In particular embodiments that may be mentioned herein, the polyimide may have a repeating unit of formula I:

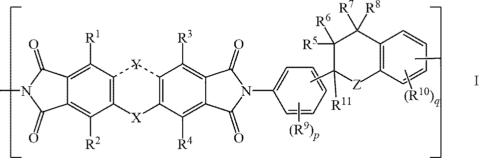

each of R¹ to R⁸ and R¹¹ is independently selected from H, C₁₋₃ alkyl, C₁₋₃ haloalkyl and halo;
X is a bond or is —C(═O)—;
Y is absent or is —C(═O)—; or
X and Y are each absent so as to form a fused aromatic structure of formula (Ia):

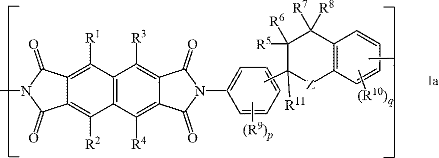

each R⁹, when present, is independently selected from the group comprising: C₁₋₃ alkyl, C₁₋₃ haloalkyl and halo;
each R¹⁰, when present, is independently selected from the group comprising: C₁₋₃ alkyl, C₁₋₃ haloalkyl and halo;
Z is selected from the group comprising: a bond and —C(R¹)₂—;
p is from 0 to 4; and
q is from 0 to 3.

In further embodiments that may be mentioned herein, a suitable polyimide that may be mentioned is one that has a repeating unit of formula II:

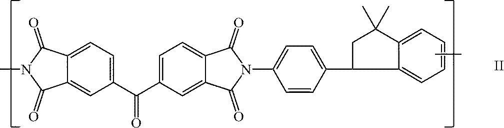

Any suitable inorganic material may be used herein. For example, the inorganic material may be selected from one or more of a metal organic framework (MOF), carbon nanotubes, zeolites, titanium dioxide (TiO₂), nanoalumina, silica nanoparticles, silver nanoparticles, and a graphene oxide. More particularly, the inorganic material may be a MOF. Without wishing to be bound by theory, it is believed that the presence of a MOF may improve both the resistance and flux in mixed matrix membrane.

When the inorganic material is a MOF, it may have one or more of the following properties:
(a) a porosity of greater than or equal to 50% of the MOF crystal volume, such as from 55 to 80%, such as from 60 to 70%;
(b) a surface area of from 1,000 to 10,000 m²/g, such as from 2,000 to 8,000 m²/g, such as from 4,000 to 6,000 m²/g;
(c) a diameter of the particle of from 10 to 1,000 nm, such as from 25 to 500 nm, such as from 50 to 150 nm.

Examples of suitable MOFs that may be used as the inorganic material includes, but is not limited to, one or more of a UiO-66 MOF (e.g. UiO-66, UiO-66-COOH, UiO-66-F₄ and UiO-66-NH₂), a MIL-53(AI) MOF, a ZIF-8 MOF, and a HKUST-1 MOF. In particular embodiments, suitable MOFs that may be used as the inorganic material that may be mentioned herein include, but is not limited to, one or more of a UiO-66 MOF (e.g. UiO-66, UiO-66-F₄ and UiO-66-NH₂), a MIL-53(AI) MOF, a ZIF-8 MOF, and a HKUST-1 MOF.

The membrane may include any suitable amount of the polyimide polymeric matrix material and the inorganic material. For example, the weight to weight ratio of the polyimide polymeric matrix material to the inorganic material may be from 100:1 to 1000:1, such as from 100:1 to 25:1, such as from 150:1 to 200:1, such as 180:1.

As will be appreciated, in order to be solvent-resistant, the entirety of the polyimide polymeric matrix material may need to have undergone some degree of crosslinking, so that the polymer strands do no solubilise in an organic solvent. As such, the polymeric matrix material may be partially crosslinked by a diamine (e.g. 1,6-hexanediamine), optionally wherein the polymeric matrix material is a polyimide polymeric matrix material is partially crosslinked by a diamine (e.g. 1,6-hexanediamine).

“Partially crosslinked”, when used herein is intended to refer to the presence of crosslinks generated throughout the solvent-resistant polymeric matrix by a second crosslinking agent that makes use of some, but not all, of the available crosslinking sites in the polymeric matrix. This allows a further crosslinking agent to react with available sites in the first region of the polymeric matrix to generate the desired OSN or OSRO membrane. This partial crosslinking can be generated by the use of a first crosslinking reagent when the polymeric matrix is first generated. The subsequent further crosslinking of the first region to generate the OSN or OSRO membrane may then be generated by the use of vapour phase crosslinking as discussed in more detail herein. Any suitable material may be used to generate the crosslinks, such as a polyamine as defined herein.

As will be appreciated, it is difficult to fully quantify the degree of crosslinking in this partial crosslinking step. However, if the material is fully crosslinked, then it would not be possible for the first region to be subjected to further crosslinking. As such, the degree of crosslinking has to leave sufficient free sites to enable a second round of crosslinking to occur in the first region of the mixed matrix material. For example, the degree of partial crosslinking may be less than 90%, less than 80%, less than 70%, less than 60%, less than 50%, less than 40%, less than 30%, less than 20%, less than 10%, less than 5%, less than 2.5%, less than 2%, or less than 1% of the available crosslinking sites. As will be appreciated, the only requirement for this partial crosslinking is that there are sufficient crosslinks present throughout the polymeric matrix to render it solvent-resistant.

Any suitable crosslinking agent may be used herein. For example, the crosslinking agent may be selected from one or more of the group consisting of a polyamine, and a hydrazine. In particular embodiments disclosed herein, the crosslinking agent may be a polyamine.

When used herein, the term “polyamine” refers to any compound having two or more primary amine groups. In certain embodiments of the invention that may be mentioned herein, at least two of the at least two primary amino groups may be separated by seven or more atoms (for the avoidance of doubt not including the nitrogen atoms of the amino groups). For example, at least two of the at least two primary amino groups may be separated by ten or more atoms.

Examples of suitable polyamines that may be mentioned herein include, but are not limited to ethylenediamine (EDA), trimethylamine (TEA) or, more particularly, tris(2-aminoethyl)amine (TAEA), N,N′-bis(3-aminopropyl)-1,3-propanediamine (APPD), 1,4-bis(3-aminopropyl) piperazine (BAPP), and polyethylenimine (PEI), optionally wherein the polyamine is selected from one or more of the group consisting of N,N′-bis(3-aminopropyl)-1,3-propanediamine (APPD), 1,4-bis(3-aminopropyl) piperazine (BAPP), polyethylenimine (PEI), and combinations thereof. In particular embodiments that may be mentioned herein, the polyamine may be selected from APPD and/or PEI. Without wishing to be bound by theory, it is believed that APPD and PEI may be effective in generating smaller pores in the dense-selective layer that is formed atop of the membrane surface during the vapour phase crosslinking process. As such, narrow pore size distributions and the ability for precise molecule separation can be achieved for the membranes. PEI when used herein may have any suitable weight average molecular weight. For example, PEI in embodiments of the invention that may be mentioned herein may have a weight average molecular weight of from 100 to 1,000 g mol⁻¹., such as 800 g mol⁻¹.

The new and simple vapour phase crosslinking method disclosed herein has an advantage of localizing the crosslinking region and thus offers a higher crosslinking efficiency and shorter reaction time (less than 30 min) than conventional chemical crosslinking methods. It is also capable of inducing an ultrathin dense-selective layer with high permeance and selectivity in the membrane surface region. In addition, the vapour phase crosslinking method mentioned herein is environmentally-friendly as the vapour phase crosslinking method uses a small amount of amine vapour and waste production is reduced. At the same time, the polyamines used herein are recyclable by cooling reactive agents.

When weight average molecular weight of a polymer is mentioned herein, it may be measured using light scattering. When the number average molecular weight of a polymer is mentioned herein, it may be measured using gel permeation chromatography.

The first region may have any suitable mean effective pore size. For example, the first region may have a mean effective pore size of from 0.4 to 1.2 nm, such as from 0.5 to 1.13 nm, such as 0.6 to 0.9 nm.

The mixed matrix material disclosed herein may have any suitable thickness. For example, the mixed matrix material may have a thickness of from 20 to 150 µm, such as from 25 to 110 µm.

As noted, the mixed matrix material is formed from a first and second region. The first region differs from the second region in that it has been subjected to (additional) crosslinking. The first region comprises one of the surfaces of the membrane and extends from this surface to a depth part-way through the membrane, meaning that the first region has a thickness. Any suitable thicknesses for the first region (provided it is not the same as the thickness of the mixed matrix material) may be used herein. For example, the first region may have a thickness of from 100 to 5,000 nm, such as from 200 to 3,500 nm, such as from 300 to 3,000 nm, such as from 500 to 2,500 nm.

For the avoidance of doubt, it is explicitly contemplated that where a number of numerical ranges related to the same feature are cited herein, that the end points for each range are intended to be combined in any order to provide further contemplated (and implicitly disclosed) ranges. Thus, in relation to the related numerical ranges, relating to thickness of the first region mentioned below (a thickness of from 100 to 5,000 nm, such as from 200 to 3,500 nm, such as from 300 to 3,000 nm, such as from 500 to 2,500 nm), there is disclosed a thickness of:
from 100 to 200 nm, from 100 to 300 nm, from 100 to 500 nm, from 100 to 2,500 nm, from 100 to 3,000 nm, from 100 to 3,500 nm, from 100 to 5,000 nm;
from 200 to 300 nm, from 200 to 500 nm, from 200 to 2,500 nm, from 200 to 3,000 nm, from 200 to 3,500 nm, from 200 to 5,000 nm;
from 300 to 500 nm, from 300 to 2,500 nm, from 300 to 3,000 nm, from 300 to 3,500 nm, from 300 to 5,000 nm;
from 500 to 2,500 nm, from 500 to 3,000 nm, from 500 to 3,500 nm, from 500 to 5,000 nm; from 2,500 to 3,000 nm, from 2,500 to 3,500 nm, from 2,500 to 5,000 nm;
from 3,000 to 3,500 nm, from 3,000 to 5,000 nm; and
from 3,500 nm to 5,000 nm.

As an additional example, the following possible ranges for the mean effective pore size of the first region are explicitly contemplated:
from 0.2 to 0.4 nm, from 0.2 to 0.5 nm, from 0.2 to 0.6 nm, from 0.2 to 0.9 nm, from 0.2 to 1.0 nm, from 0.2 to 1.13 nm, from 0.2 to 1.2 nm, from 0.2 to 2 nm;
from 0.4 to 0.5 nm, from 0.4 to 0.6 nm, from 0.4 to 0.9 nm, from 0.4 to 1.0 nm, from 0.4 to 1.13 nm, from 0.4 to 1.2 nm, from 0.4 to 2 nm;
from 0.5 to 0.6 nm, from 0.5 to 0.9 nm, from 0.5 to 1.0 nm, from 0.5 to 1.13 nm, from 0.5 to 1.2 nm, from 0.5 to 2 nm;
from 0.6 to 0.9 nm, from 0.6 to 1.0 nm, from 0.6 to 1.13 nm, from 0.6 to 1.2 nm, from 0.6 to 2 nm;
from 0.9 to 1.0 nm, from 0.9 to 1.13 nm, from 0.9 to 1.2 nm, from 0.9 to 2 nm;
from 1.0 to 1.13 nm, from 1.0 to 1.2 nm, from 1.0 to 2 nm;
from 1.13 to 1.2 nm, from 1.13 to 2 nm; and
from 1.2 to 2 nm.

When used herein, the diameter of the nanoparticles may be measured by FESEM. For example, the diameter of the nanoparticles may be calculated based on a statistical analysis of their FESEM images.

In certain embodiments of the invention, the membrane may further comprise a non-woven substrate. In such embodiments, the non-woven substrate may be formed from any suitable material. For example, the non-woven substrate may be formed from one or more of the group consisting of a polyester (PET) and a polypropylene (PP). For example, the non-woven substrate may be formed from polyethylene terephthalate.

The organic solvent nanofiltration (OSN) membrane and/or an organic solvent reverse osmosis (OSRO) membrane may be in any suitable shape or form for use in its intended purpose. For example, it may be presented as a flat membrane or it may be presented as a hollow fibre membrane. As will be appreciated, if the OSN and/or an organic solvent reverse osmosis (OSRO) membrane is presented as a hollow fibre membrane, it will not require a non-woven substrate.

The membranes disclosed herein may display a molecular weight cut-off value, thereby allowing the separation of materials from one another. For example, the membrane may display a molecular weight cut-off value of from 100 to 500 Daltons. Additionally, in certain embodiments, a rejection rate for a material having a molecular weight that is at least 10% above the molecular weight cut-off value of the membrane may be at least 90%. As will be appreciated, a molecular weight cut-off value of from 400 to 500 Daltons may be particularly suited to an OSN membrane.

A molecular weight cut-off value for an OSRO membrane may be less than or equal to 200 Daltons. For example, the molecular weight cut-off value for an OSRO membrane may be from 100 to 200 Daltons. Reference: Cuijing Liu, Guanying Dong, Toshinori Tsuru, Hideto Matsuyama, Organic solvent reverse osmosis membranes for organic liquid mixture separation: A review, Journal of Membrane Science, Volume 620, 2021, p118882. Additionally, in certain embodiments, a rejection rate for a material having a molecular weight that is at least 10% above the molecular weight cut-off value of the membrane may be at least 90%.

The molecular weight cut-off values obtained may be influenced by the selected crosslinking agent. For example, when one uses a mesoporous membrane support with a relatively small effective pore size already (e.g. from 5 to 15 nm, such as from 6 to 10 nm, such as 6.92 nm), then a linear polyamine with less distance between the active primary amine groups may result in a material with a lower molecular weight cut-off value, while one with a greater distance may have a higher molecular weight cut off value. The opposite may be true if the mesoporous membrane support has a relatively large effective pore size (e.g. from 18 to 30 nm, such as 20 nm). In this case, a linear polyamine with a greater distance between the active primary amine groups may result in a material with a lower molecular weight cut-off value than a polyamine with less distance between the primary amine groups. This reversal may occur because the pore of the support is too large, meaning that a polyamine with less distance between the active primary amine groups cannot generate effective crosslinking between polymer matrixes. That is, a polyamine which has a distance between the primary amine groups that is less than the size of the pore may not be able to effectively bridge the entire pore, leading to a larges pore size that one would expect.

The membranes disclosed herein may be suitable for use in nanofiltration applications using one or more solvents selected from two or more of the solvent classes: polar protic, polar aprotic and non-polar organic solvents. For example, the membrane is suitable for use in nanofiltration applications using one or more solvents selected from polar protic, polar aprotic and non-polar organic solvents. As will be appreciated, the membrane is suitable for use in a wide variety of solvents.

The membranes disclosed herein may have a pure solvent permeance value of from 0.1 to 4 L m⁻² h⁻¹ bar⁻¹. In particular embodiments of the invention that may be mentioned herein, the membrane may have one or more of the following pure solvent permeance values:
(a) from 2 - 4 L m⁻² h⁻¹ bar⁻¹ for methanol;
(b) from 0.5 - 1.5 L m⁻² h⁻¹ bar⁻¹ for isopropanol (IPA);
(c) from 0.6 - 4 L m⁻² h⁻¹ bar⁻¹ for hexane;
(d) from 0.9 - 1.5 L m⁻² h⁻¹ bar⁻¹ for toluene;
(e) from 0.8-1.3 L m⁻² h⁻¹ bar⁻¹ for tetrahydrofuran (THF);
(f) from 0.2-1 L m⁻² h⁻¹ bar⁻¹ for dimethylformamide (DMF); and
(g) from 1 -2 L m⁻² h⁻¹ bar⁻¹ for ethanol.

More particularly, the membrane may have one or more of the following pure solvent permeance values:
(a) from 2 -4 L m⁻² h⁻¹ bar⁻¹ for methanol;
(b) from 0.5 - 1.5 L m⁻² h⁻¹ bar⁻¹ for isopropanol (IPA);
(c) from 0.6 - 4 L m⁻² h⁻¹ bar⁻¹ for hexane;
(d) from 0.9 - 1.5 L m⁻² h⁻¹ bar⁻¹ for toluene;
(e) from 0.8-1.3 L m⁻² h⁻¹ bar⁻¹ for tetrahydrofuran (THF); and
(f) from 0.2-1 L m⁻² h⁻¹ bar⁻¹ for dimethylformamide (DMF).

As will be appreciated, the membranes disclosed herein may be used in nanofiltration. As such, there is also disclosed a method of nanofiltration using an organic solvent nanofiltration (OSN) membrane as described hereinbefore, comprising the steps of
(a) providing a solution comprising a first compound having a first molecular weight and a second compound having a second molecular weight; and
(b) subjecting the solution to nanofiltration using an OSN membrane as described in any one of Clauses 1 to 25, such that the first and second compounds are separated from one another, wherein
   the first molecular weight is lower than the second molecular weight and the OSN membrane has a molecular weight cut-off that prevents the second compound from passing through the membrane, thereby separating the first and second compounds. Further details of the method of use may be obtained from the examples section below and by analogy to the methods disclosed therein.

In certain embodiments of the invention, the first and second compounds may be solvents and so the solution does not need a separate solvent. As such, the nanofiltration may relate to solvent-solvent extraction. Alternatively, the method may be one in which the solution may further comprise an organic solvent and the first and second compounds are not organic solvents. In this instance, the nanofiltration may relate to the separation of two compounds.

Also disclosed herein is a method of forming an organic solvent nanofiltration (OSN) membrane and/or an organic solvent reverse osmosis (OSRO) membrane comprising a mixed matrix material as described hereinbefore, wherein the method comprises the steps of:
(a) providing a material comprising:
   a partially crosslinked solvent-resistant polymeric matrix with functional groups suitable for crosslinking; and
   an inorganic material homogeneously dispersed throughout the polymeric matrix, where the material has a first region comprising a surface of the material and a second region; and
(b) exposing the first region of the material to a vapour comprising a crosslinking agent that generates further crosslinks in said first region to provide the organic solvent nanofiltration (OSN) membrane. Further details of the method of manufacture may be obtained from the examples section below and by analogy to the methods disclosed therein.

Also disclosed herein is a method of nanofiltration using an organic solvent reverse osmosis (OSRO) membrane as described hereinbefore, comprising the steps of
(a) providing a solution comprising a first solvent having a first molecular weight and a second solvent having a second molecular weight; and
(b) subjecting the solution to nanofiltration using an OSRO membrane as described in any one of Clauses 1 to 25, such that the first and second solvents are separated from one another, wherein
   the first molecular weight is lower than the second molecular weight and the OSRO membrane has a molecular weight cut-off that prevents the second solvent from passing through the membrane, thereby separating the first and second solvents. Further details of the method of use may be obtained from the examples section below and by analogy to the methods disclosed therein.

Also disclosed herein is a method of forming an organic solvent reverse osmosis (OSRO) membrane comprising a mixed matrix material as described hereinbefore, wherein the method comprises the steps of:
(a) providing a material comprising:
   a partially crosslinked solvent-resistant polymeric matrix with functional groups suitable for crosslinking; and
   an inorganic material homogeneously dispersed throughout the polymeric matrix, where the material has a first region comprising a surface of the material and a second region; and
(b) exposing the first region of the material to a vapour comprising a crosslinking agent that generates further crosslinks in said first region to provide the organic solvent reverse osmosis (OSRO) membrane. Further details of the method of manufacture may be obtained from the examples section below and by analogy to the methods disclosed therein.

Further aspects and embodiments of the invention will now be discussed by reference to the following non-limiting examples.

### EXAMPLES

### Materials

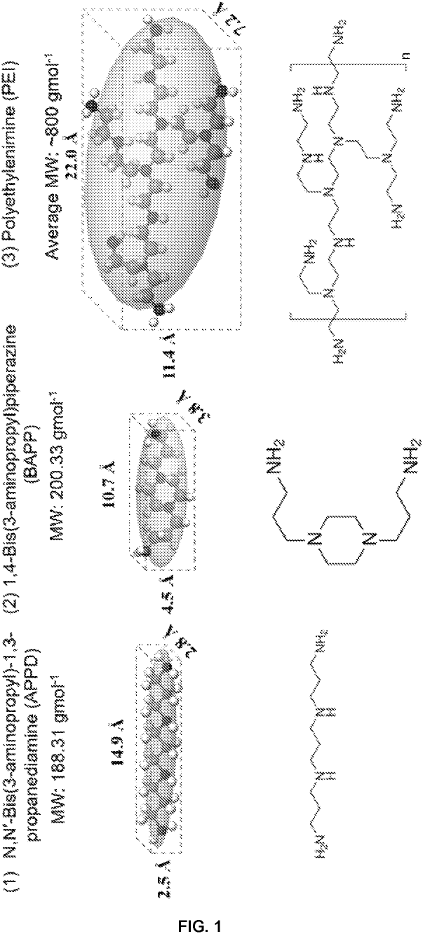

The commercial Matrimid® 5218 polymer was supplied by Vantico Inc. (USA). The polybenzimidazole (PBI) dope was provided by PBI Performance Products Inc. in a solution with a composition of 26 wt% PBI and 74 wt% dimethylacetamide (DMAc). The PET non-woven fabrics were provided by SEFAR (Switzerland). Analytical grade N-methyl-2-pyrrolidinone (NMP), diethylene glycol (DEG), dimethylformamide (DMF), propanol, 2-aminoterephthalic acid (ACD) and acetic acid (AA) were acquired from Merck (Germany). High-performance liquid chromatography grade tetrahydrofuran (THF), dimethylacetamide (DMAc), methanol (MeOH), ethanol (EtOH), isopropanol (IPA), toluene and n-hexane were purchased from Fisher Scientific (UK). 1,6-hexanediamine (HDA,> 98%), glycerol, α, α′-dibromo-p-xylene (DBX, 97%), lithium chloride (LiCI), tris-(2-aminoethyl) amine (TAEA, 96%) and polyvinylpyrrolidone (PVP, average MW 15 kDa) were ordered from Sigma-Aldrich (Singapore). Zirconium chloride (ZrCl₄) was purchased from TCI (Japan). The crosslinking reagents, N, N′-Bis(3-aminopropyl)-1,3-propanediamine (APPD), 1,4-Bis(3-aminopropyl) piperazine (BAPP) and polyethylenimine (PEI), with an average MW of approximately 800 g mol⁻¹ (FIG. 1) were supplied by Sigma-Aldrich (Singapore). Neutral polyethylene glycols (PEGs) and polyethylene oxides (PEOs) with different MW of 400, 600, 1,000, 2,000, 4,000, 12,000, 20,000 and 35,000 g mol⁻¹ from Merck (Singapore) were used as markers to characterize the mean pore size and pore size distribution of the membranes. Rose Bengal (RB, MW = 1018 g mol⁻¹), Brilliant Blue R (BBR, MW = 826 gmol⁻¹), Eosin Y (EY, MW = 648 g mol-1), Tetracycline (TC, MW = 444 g mol⁻¹), and Sudan IV (SI, MW = 380 g mol⁻¹) were obtained from Sigma-Aldrich (Singapore) as solutes in OSN tests. The deionized (DI) water was produced by a Milli-Q unit (Millipore). A slow curing epoxy resin (EP231, Kuo Sen, Taiwan) was used for module potting. All chemicals in this study were used as received without further purification. All chemicals were used without further purification.

### Analytical Techniques

### Field Emission Scanning Electron Microscopy (FESEM)

Field emission scanning electron microscopy (FESEM, JEOL JSM-6700F) was employed to observe the morphologies of membrane surfaces and cross-sections. For membranes associated with PET nonwoven supports, the fabric layers were removed carefully by peeling them away from the polymeric layers. All FESEM samples were coated with platinum by an ion sputtering device (JEOL JFC-1300E). For characterization of membrane cross-sections, vacuum dried samples were fractured prior in liquid nitrogen before the platinum coating.

### Contact Angle Measurements

The contact angles of the solvents on the membrane surfaces were measured by an optical contact angle drop-meter (DataPhysics, OCA25, Germany) at room temperature with a relative humidity of roughly 40%. Ten random locations were selected for each sample with a size of 5 cm × 1 cm and three independent membranes were examined for each condition.

### X-ray Photoelectron Spectroscopy (XPS)

XPS (Kratos AXIS Ultra^{DLD}) equipped with a monochromatized AI Kα X-ray source (1486.71 eV, 5 mA, 15 kV) was used to analyze the chemical composition on membrane surfaces.

### Doppler Broadening Energy Spectroscopy

The Doppler broadening energy spectroscopy of an in-house positron annihilation lifetime spectroscope was applied to investigate the free volume and thickness variation of skin layers on the pristine MMM substrates and VPC modified membranes. The R parameter was measured as a function of positron incident energy from 0 to 30 keV using the monoenergy slow positron beam under an ultra-high vacuum of ~10⁻⁷ Torr. It was defined as the ratio of the complete counts from ortho-positronium (o-Ps) 3γ annihilation to the total counts from the 511 keV peak region (due to 2 y annihilation) (Y. C. Jean, P. E. Mallon & D. M. Schrader, Principles and Applications of Positron & Positronium Chemistry, World Scientific Publishing Co. Pte. Ltd., Singapore, 2003) to qualitatively measure the free volume and the existence of pores in nm to µm sizes.

The mean depth Z of the membranes was evaluated using Eq. (1) as follows (Y. C. Jean, P. E. Mallon & D. M. Schrader, Principles and Applications of Positron & Positronium Chemistry, World Scientific Publishing Co. Pte. Ltd., Singapore, 2003; and H. Chen et al., Macromolecules 2007, 40, 7542-7557):

$$Z \left({E}_{+}\right) = \left(\frac{40}{ρ}\right) \times {E}_{+}^{1.6} \qquad \text{(1)}$$

where Zrefers to the depth (nm), ρ denotes the material density (g cm⁻³), and E₊ is the incident positron energy (keV).

### Measurements of Pore Size Distribution and Mean Effective Pore Size

The mean pore size and its distribution of the HDA-crosslinked MMM substrates and VPC modified hybrid membranes in aqueous systems were examined by using a lab-scale dead-end permeation cell (P. Aimar et al., J. Membr. Sci. 1990, 54, 321-338; S. Singh et al., J. Membr. Sci. 1998, 142, 111-127; and B. Van der Bruggen & C. Vandecasteele, Water Research 2002, 36, 1360-1368). The solutes PEG and PEO with various molecular weights of 400 - 35,000 g mol⁻¹ were used to prepare feed solutions of 200 ppm in DI water for filtration tests. The solute concentrations in the feed (C_{f}) and permeate (Cₚ) were measured by a total organic carbon analyzer (TOC ASI-5000A, Shimadzu, Japan). The effective solute rejection coefficient R (%) was then calculated as:

$$R = \left(1 - \frac{{C}_{P}}{{C}_{F}}\right) \times 100 \% \qquad \text{(2)}$$

The correlation between Stoke diameters (dₛ, m) of PEG and PEO solutes and their molecular weights (g mol⁻¹) could be written by Eq. (3) and (4) (P. Aimar et al., J. Membr. Sci. 1990, 54, 321-338; S. Singh et al., J. Membr. Sci. 1998, 142, 111-127; and B. Van der Bruggen & C. Vandecasteele, Water Research 2002, 36, 1360-1368),

$${d}_{s} = 33.46 \times {10}^{- 12} \times M {w}^{0.557} \left(M w \leq 35 , 000\right) \qquad \text{(3)}$$

$${d}_{s} = 20.88 \times {10}^{- 12} \times M {w}^{0.587} \left(M w \geq 100 , 000\right) \qquad \text{(4)}$$

Thus, the pore size distribution can be expressed by the following probability density function as Eq. (5) (P. Aimar et al., J. Membr. Sci. 1990, 54, 321-338; S. Singh et al., J. Membr. Sci. 1998, 142, 111-127; B. Van der Bruggen & C. Vandecasteele, Water Research 2002, 36, 1360-1368; and N. Widjojo et al., J. Membr. Sci. 2011, 383, 214-223):

$$\frac{d R \left({d}_{p}\right)}{d {d}_{p}} = \frac{1}{{d}_{p} ln {σ}_{p} \sqrt{2 π}} exp \left[- \frac{{\left(ln {d}_{p} - ln {μ}_{p}\right)}^{2}}{2 {\left(ln {σ}_{p}\right)}^{2}}\right] \qquad \text{(5)}$$

where dₚ is the pore diameter (nm), µₚ is the mean effective pore size (nm) at R =50% and σₚ is determined by the ratio of pore diameter at R = 84.13% over the one at R = 50%.

Attenuated Total Reflection-Fourier- Transform Infrared Spectroscopy (ATR-FTIR) ATR-FTIR was carried out on Bio-Rad TFS-3500 FTIR under an attenuated total reflectance mode over a wavenumber range of 400 - 4000 cm⁻¹.

### Example 1. Synthesis of UiO-66-NH₂ Nanoparticles

The nano-size amine-functionalized MOF particles (i.e. UiO-66-NH₂) were synthesized by the solvothermal method (D. Ma et al., Ind. Eng. Chem. Res. 2017, 56, 12773-12782).

The reacting solution was prepared by dissolving ZrCl₄ (80 mg), ACD (62 mg) and AA (0.60 mL) in DMF (20 mL). It was mixed and heated to 120° C. for 24 h under stirring. Afterwards, the precipitates were separated by centrifugation at 12,000 revolutions per minute (rpm), washed with DMF and MeOH for several times via centrifugation and sonication, respectively. Lastly, the obtained white particles were vacuum-dried at 200° C. overnight. They had an average particle size of around 150 nm.

### Example 2. Fabrication of Crosslinked MMM Substrates

### Pristine MMM Substrates

An 18 wt% polyimide dope solution consisting of Matrimid® 5218/NMP/THF/DEG at a weight ratio of 18/66/6/10 and UiO-66-NH₂ particles of 0.1 wt% based on the Matrimid® mass was stirred at 50° C. for 12 h, followed by deaerating for another day at room temperature. The 0.1 wt% UiO-66-NH₂ with a particle size of 150 nm was chosen because the resultant substrate had the most balanced performance for OSN in our previous study (Z. F. Gao et al., J. Membr. Sci. 2019, 574, 124-135). The homogenous dope solution was cast onto a glass plate mounted on the glass plate by a casting knife at a speed of 0.015 ms⁻¹. The gap thickness of the casting knife ranged from 100 µm to 150 µm. After casting, the glass plate was immediately immersed into an IPA or water bath which contained 2 wt% HDA under 4° C. for 12 h. Subsequently, the membranes were peeled off and transferred to a clean IPA or water bath at room temperature for two days to remove the residual solvents. Then, the wet membrane substrates were solvent exchanged with IPA and n-hexane thrice, each time lasting for 30 min, then dried in air and annealed in an oven at 120° C. overnight before further usage.

Table 1 displays the identifications (IDs) of various substrates, casting knife gap, coagulant bath, temperatures and pressures.

TABLE 1

List of membranes and fabrication conditions.

| Membrane ID | Castin knife gap (µm) | Casting support | Coagulation bath | VPC agent | Temperature ◦C) | Pressure (bar) | Treatment ducation min) |
|---|---|---|---|---|---|---|---|
| S1 | 150 | - | 2 wt% HDA in IPA | - | - | - | - |
| S2-N | 150 | Polyester non-woven | 2 wt% HDA in IPA | - | - | - | - |
| S3-N | 150 | Polyester non-woven | 2 wt% HDA in water | - | - | - | - |
| S4-N | 100 | Polyester non-woven | 2 wt% HDA in water | - | - | - | - |
| S1-APPD-10 | 150 | - | 2 wt% HDA in IPA | APPD | 120 | 1.0 | 10 |
| S1-APPD-20 | 150 | - | 2 wt% HDA in IPA | APPD | 120 | 1.0 | 20 |
| S1-APPD-30 | 150 | - | 2 wt% HDA in IPA | APPD | 120 | 1.0 | 30 |
| S1-APPD-45 | 150 | - | 2 wt% HDA in IPA | APPD | 120 | 1.0 | 45 |
| S1-BAPP-10 | 150 | - | 2 wt% HDA in IPA | BAPP | 120 | 1.0 | 10 |
| S1-BAPP-20 | 150 | - | 2 wt% HDA in IPA | BAPP | 120 | 1.0 | 20 |
| S1-BAPP-30 | 150 | - | 2 wt% HDA in IPA | BAPP | 120 | 1.0 | 30 |
| S1-BAPP-45 | 150 | - | 2 wt% HDA in IPA | BAPP | 120 | 1.0 | 45 |
| S1-PEI-10 | 150 | - | 2 wt% HDA in IPA | PEI | 140 | 0.1 | 10 |
| S1-PEI-20 | 150 | - | 2 wt% HDA in IPA | PEI | 140 | 0.1 | 20 |
| S1-PEI-30 | 150 | - | 2 wt% HDA in IPA | PEI | 140 | 0.1 | 30 |
| S1-PEI-45 | 150 | - | 2 wt% HDA in IPA | PEI | 140 | 0.1 | 45 |
| S4-N-APPD-20 | 100 | Polyester non-woven | 2 wt% HDA in water | APPD | 120 | 1.0 | 20 |
| S4-N-BAPP-20 | 100 | Polyester non-woven | 2 wt% HDA in water | BAPP | 120 | 1.0 | 20 |
| S4-N-PEP-30 | 100 | Polyester non-woven | 2 wt% HDA in water | PEI | 120 | 0.1 | 20 |

### MMM/PET Non-Woven Substrates

MMM/PET non-woven substrates were prepared by following the protocol for MMM substrates except the homogenous dope solution was cast onto a PET non-woven fabric (Table 1).

### Example 3. Membrane Vapour-phase Crosslinking (VPC) via Amine Vapour to Obtain VPC Modified Membranes

The VPC modification of membranes was conducted according to our previous study (Z. F. Gao et al., J. Membr. Sci. 2019, 574, 124-135).

### VPC/MMM Membranes

A petri dish containing a 20 mL solution of monomers selected from APPD, BAPP or PEI was covered by a glass plate and conditioned in an oven at the targeted temperature and pressure for 1 h. After that, a glass plate which was already mounted by a piece of the crosslinked pristine MMM substrates (prepared in Example 2) quickly replaced the original covering glass plate. The denser surface of the substrate was placed facing the vapour directly. The duration of vapour exposure varied from 0 to 45 min, followed by annealing in a vacuum oven at 120° C. overnight.

### VPC/MMM/PET Membranes

VPC/MMM/PET membranes were prepared from MMM/PET non-woven S4-N substrates by following the protocol for VPC/MMM membranes.

Table 1 displays the identifications (IDs) of VPC modified membranes and their casting conditions, crosslinker chemistry, vapour exposure durations, modification temperatures and pressures.

### Example 4. Organic Solvent Nanofiltration (OSN) Performance Tests

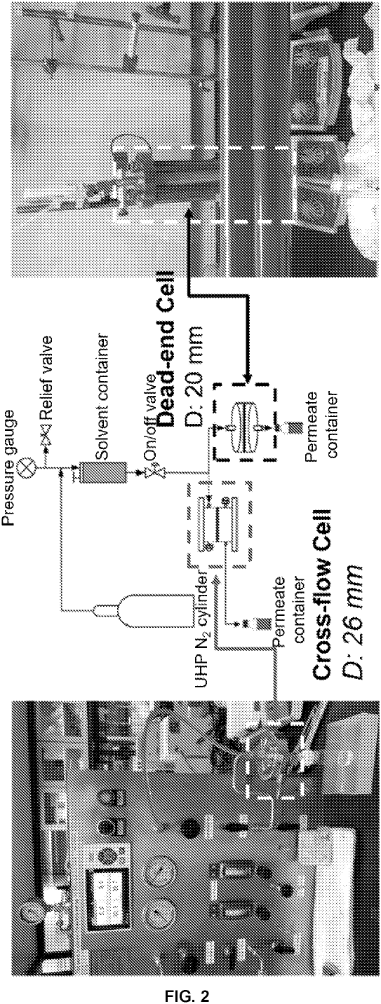

A cross-flow setup (FIG. 2) was used to test the OSN performance of the newly developed VPC/MMM/PET membranes in Example 3 because it could not only minimize the fouling effect but also mimic the industrial process (G. M. Shi et al., J. Membr. Sci. 2019, 588, 117202; and G. Mustafa et al., Water Res. 2016, 104, 242-253). Organic dyes with various MWs and shapes (Table 2) were employed in the dye-IPA system as feeds.

TABLE 2

Structures and physicochemical properties of solutes for filtration tests.

| | | | | | |
|---|---|---|---|---|---|
| Dye | Rose Bengal (RB) | Brilliant Blue R (BBR) | Eosin Y (EY) | Tetracycline (TC) | Sudan IV (SI) |
| Molecular Structure | 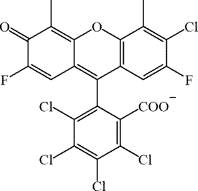 | 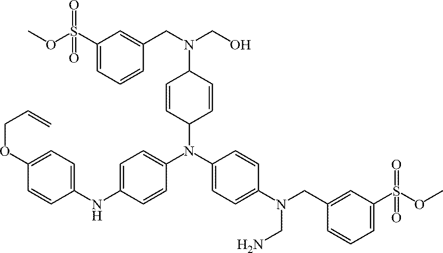 [SMILES: C=CCOc1ccc(Nc2ccc(N(c3ccc(N(CN)Cc4cccc(S(=O)(=O)OC)c4)cc3)C3C=CC(N(CO)Cc4cccc(S(=O)(=O)OC)c4)C=C3)cc2)cc1] | 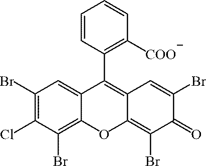 | 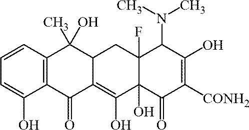 [SMILES: CN(C)C1C(O)=C(C(N)=O)C(=O)C2(O)C(O)=C3C(=O)c4c(O)cccc4C(C)(O)C3CC12F] | 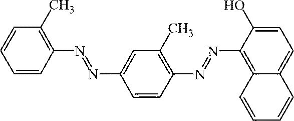 [SMILES: Cc1ccccc1/N=N/c1ccc(/N=N/c2c(O)ccc3ccccc23)c(C)c1] |
| MW (g mol⁻¹) | 1017 | 826 | 648 | 444 | 380 |
| Charge | Negative | Negative | Negative | Neutral | Neutral |

Both the dead-end and cross-flow permeation cells were employed to evaluate solvent flux and solute rejection properties, the effective membrane testing areas were 3.41 cm² and 5.41 cm², respectively. For pure solvent tests, the flux was measured after 4 h filtration conditioning period. For each type of membranes, more than three replicates were randomly chosen from independently fabricated batches and tested. The collected data were averaged and reported. The flux (Jᵥ, L m⁻² h⁻¹) and permeance (P, L m⁻² h⁻¹ bar⁻¹) were calculated by the following equations:

$${J}_{V} = \frac{{Q}_{V}}{{A}_{m}} \qquad \text{(6)}$$

$$P = \frac{{J}_{V}}{Δ P} \qquad \text{(7)}$$

where Qᵥ denotes the volumetric flow rate (L h⁻¹) of the permeate solvent, Aₘ refers to the effective filtration area (m²), and ΔP represents the transmembrane pressure (bar).

Dyes at 50 ppm in various organic solvents (i.e. MeOH, EtOH, IPA, DMF, THF, toluene and n-hexane) were prepared as feeds for OSN tests. The conditioning time was extended to 24 h for rejection tests to minimize the impact of solute adsorption. A UV-vis spectrometer (Pharo 300, Merck) was utilized to analyze the solute concentrations in the feed, permeate and retentate streams. Eq. (2) was also used to determine the solute rejection, R (%).

### Example 5. Effects of Vapour-phase Crosslinking Duration on VPC/MMMs

As IPA has high demand and widespread utilization across various industrial sectors, it was firstly used to examine the OSN performance of the VPC modified MMMs (prepared in Example 3) by following the OSN performance tests described in Example 4.

### Results and Discussion

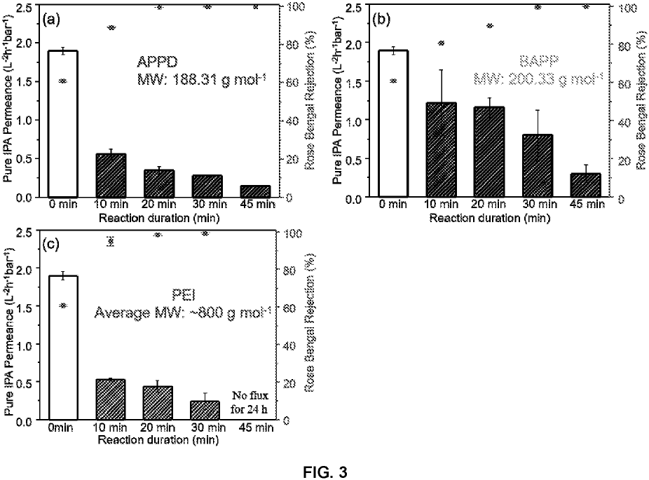

FIG. 3 displays the IPA permeance and RB rejection of VPC/MMMs as a function of crosslinking duration under a transmembrane pressure of 10.0 bar and room temperature. Beyond 20 min of VPC, the IPA permeance continued dropping while the RB rejection remained approximately constant. Therefore, the IPA permeance decreased while the RB rejection was enhanced with an increase in the duration of amine vapour exposure. The significant decline in IPA permeance may be due to the formation of a densified skin surface via amine vapour treatment.

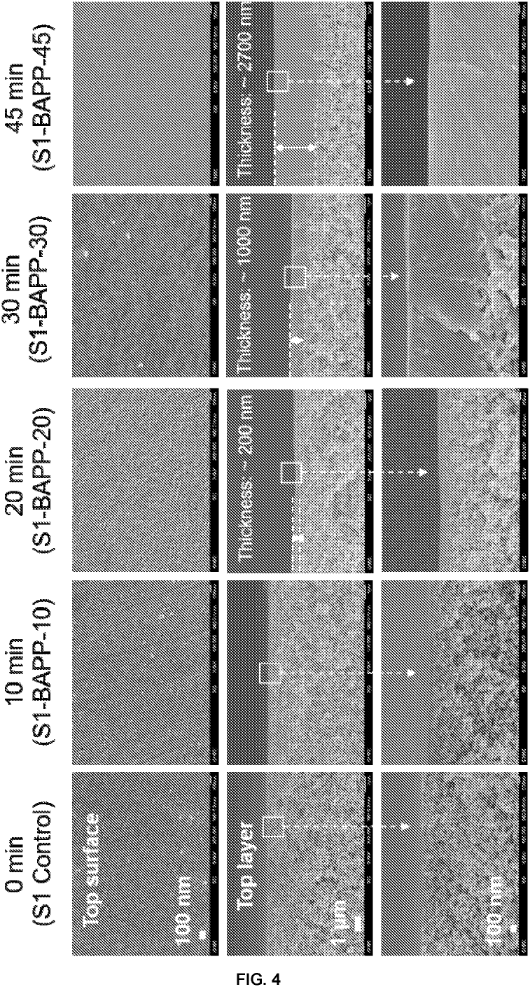

The cross-sectional FESEM images (FIG. 4) of BAPP-modified membranes taken after reaction at 120° C. and 1 atm show a thin top dense layer when the VPC duration was 10 min. The thickness of the densified layer increased dramatically once the VPC duration was over 20 min, implying a longer VPC duration resulted in a thicker dense layer.

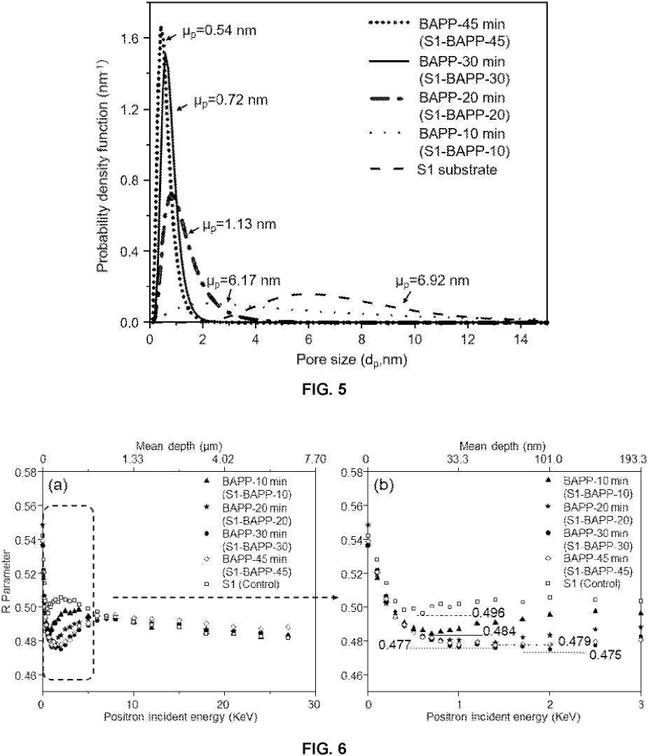

FIG. 5 compares the pore size characteristics of the BAPP-treated membranes as a function of duration for the VPC reaction. By steadily increasing the BAPP exposure duration from 0 to 10, 20, 30 and 45 min, the mean effective pore size (µₚ) decreased from 6.92 to 6.17, 1.13, 0.72 and 0.54 nm, respectively. The pore size distribution also shifted to the left and became much sharper and narrower. FIG. 6 shows the R parameter as a function of VCP duration and positron incident energy. Both FIGS. 5 and 6 are in good agreement in terms of pore size and pore size distribution.

Generally, the R parameter curve determines the evolution of pores in nm to µm sizes as a function of positron incident energy penetrating into the membrane surface (J. Zuo & T. S. Chung, J. Mater. Chem. A 2013, 1, 9814-9826). As illustrated in FIG. 6, the initial R parameter close to the surface (i.e. mean depth less than ~ 10 nm) has a decreasing trend which was caused by a relatively high amount of o-Ps 3γ annihilation at the surface due to the back diffusion and scattering of positronium (J. Zuo & T. S. Chung, J. Mater. Chem. A 2013, 1, 9814-9826). With an increase in positron incident energy, the R parameter rapidly dropped to a minimum value, which can be interpreted as the dense region of membranes (Z. Chen et al., J. Phys. Chem. C 2011, 115, 18055-18060). Then, the R value began to rise as the positron incident energy increased further, suggesting the existence of large pores in the membrane structure. This increase in R parameter indicates a change in free volume of the composite membranes and a transition from a dense layer to a porous structure (J. Zuo & T. S. Chung, J. Mater. Chem. A 2013, 1, 9814-9826). The turning points (i.e. the minimum value in the valley bottom) of R parameter curves in BAPP-treated membranes follows the order of 45 min ≥ 30 min > 20 min> 10 min, implying a consistent result with the FESEM images in FIG. 4. In addition, the thickness of the densified layer increased faster with longer VPC duration. For example, the densified layer thickness for 20 min VPC was ~200 nm, 30 min VPC was ~ 1000 nm, and 45 min VPC was ~ 2700 nm. Hence, the increased ratio of densified layer thickness in 20-30 min was 80 nm/min, and the increased ratio of densified layer thickness in 30-45 min was 113 nm/min. Therefore, the thickness of the densified layer increased faster in the 30-45 min period and clearly, the longer VPC durations yielded thicker densified areas from the top skin layer towards the transitional segment.

Although the amino crosslinking starts on the surface, the vapour-phase regents can still penetrate deeper into the bulk of the membrane (T. S. Chung, L. Shao & P. S. Tin, Macromol. Rapid Commun. 2006, 27, 998-1003). Comparing with the pore size (i.e. µₚ =6.92 nm) of the unmodified substrate, the molecular width of 4.5 Å for BAPP monomers is far smaller. Therefore, they have plenty of passageways for diffusion within the membranes with such a large pore size. Furthermore, the BAPP crosslinking procedure was carried out at a temperature of around 120° C. At this temperature, the polymer chains have more thermal energy and subsequently more segmental motion, leading to the increment of polymer inter-chain distance (A. Naderi et al., Polymer 2018, 135, 76-84). This phenomenon further confirms the ease of crosslinker penetration into the functional layer of the polymer. Besides, with the increasing duration of low-temperature heat treatment (i.e. around 100° C. and slightly above), the amidization reaction can be somewhat promoted because of further reaction between the imide groups and free amine groups (X. Y. Qiao & T. S. Chung, AIChE J. 2006, 52, 3462-3472). As a result, a higher crosslinking density was exhibited with a longer VPC duration for BAPP-modified membranes, illustrating a fast crosslinking rate forming a tighter infrastructure of both hole-filling and crosslinking network in the membranes (X. Y. Qiao & T. S. Chung, AlChE J. 2006, 52, 3462-3472).

Interestingly, the BAPP-treated membranes showed an almost opposite trend in terms of minimum R values: 10 min > 20 min ≥ 45 min ≥ 30 min, inferring a reduction in the free volume of the selective layer with an increase in VPC duration. The slight increase in the free volume of the selective layer in 45 min-modified BAPP membrane is probably attributed to the breakage of the polyimide backbones due to chain scission. Because the BAPP is excessive, the electrophilic amide groups partially exchanged with the amine groups (B. T. Low et al., Macromolecules 2008, 41, 1297-1309). Besides that, the minimum R values of BAPP-20, -30 and -45 min membranes gradually became closer, indicating that a reaction duration of 20 min or longer leads to lower free volumes and comparable porosities in the modified top layers.

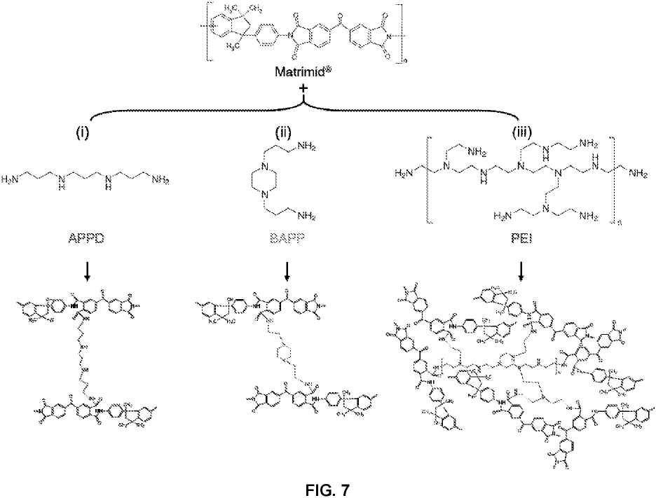

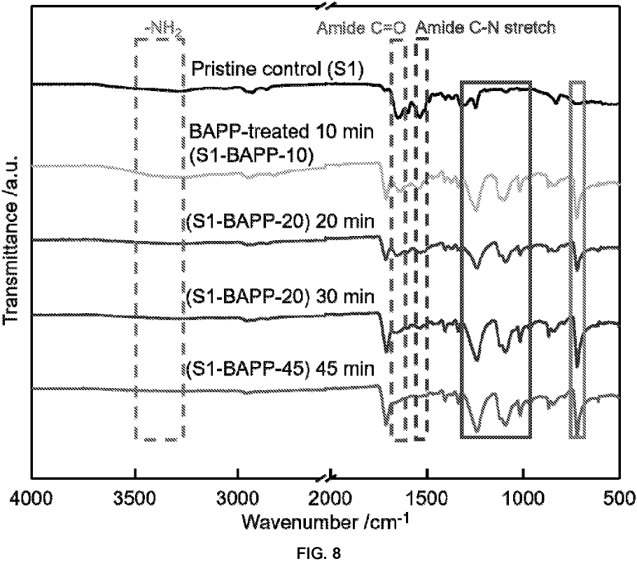

Theoretically, the narrow pore size and small porosity of VPC/MMMs are caused by a high degree of post-polymerization between vapour monomers and hybrid polymers. FIG. 7 illustrates the possible crosslinking mechanisms between the Matrimid® polymer and the three crosslinkers. The reactions between BAPP and the membrane substrate (i.e. HDA pre-crosslinked Matrimid®) were confirmed by ATR-FTIR spectra. The strong absorption at peaks of 1250 -1020 cm⁻¹ (i.e. C-N stretches of aliphatic amines) and 670 -760 cm⁻¹ (i.e. N-H wags of secondary amines and C-N stretches of tertiary amines) in FIG. 8 reveal the success of the BAPP-induced crosslinking modification (X. Y. Qiao & T. S. Chung, AlChE J. 2006, 52, 3462-3472; B.T. Low et al., Macromolecules 2008, 41, 1297-1309; B.C. Smith, Spectroscopy 2019, 34, 22-25; J. Gao et al., J. Membr. Sci. 2014, 452, 300-310; and K. Gupta, B. S. Yadav & S. Chand, Int. J. Adv. Res. Sci. Eng. 2017, 6, 300-310).

Intriguingly, with a BAPP vapour treatment of 10 min, peaks in the range of 3250 -3500 cm⁻¹ (i.e. N-H stretches of primary amines, representing unreacted free amines (D. W. Mangindaan et al., Chem. Eng. Sci. 2015, 122, 14-23; and T. Yoshioka et al., J. Appl. Polym. Sci. 2017, 44569, 1-9)) and peak intensities at 1546 and 1643 cm⁻¹ were observed, inferring that the amide groups are visible (S. P. Sun et al., AIChE J. 2014, 60, 3623-3633). These peaks suggest a partial conversion of the free amine groups. Thus, the dominant reaction in this stage is the one-end grafting of BAPP molecules (Z. F. Gao et al., J. Membr. Sci. 2019, 574, 124-135; and P. Sysel et al., Polym Eng. & Sci. 2017, 57, 1367-1373). When the vapour exposure duration was increased to 30 and 45 min, the peaks denoting primary amines and the amide groups became weaker and finally vanished, while the C-N stretching and N-H waging peaks became stronger. These findings confirm that more BAPP molecules are deposited on the substrates and the governing reactions shift from one-end grafting to fully crosslinking modification (S. Japip et al., J. Membr. Sci. 2016, 497, 248-258; Z. F. Gao et al., J. Membr. Sci. 2019, 574, 124-135; and D. W. Mangindaan et al., Chem. Eng. Sci. 2015, 122, 14-23).

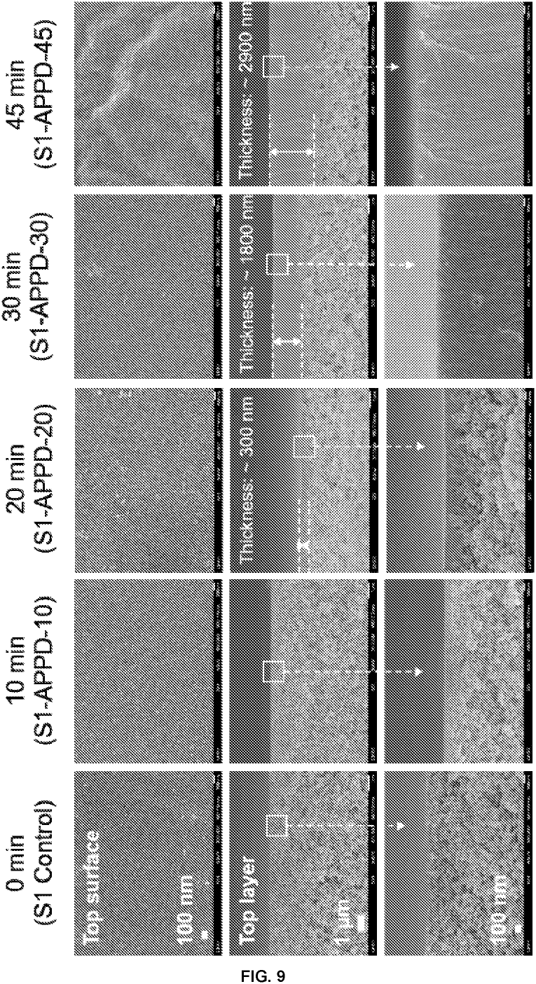

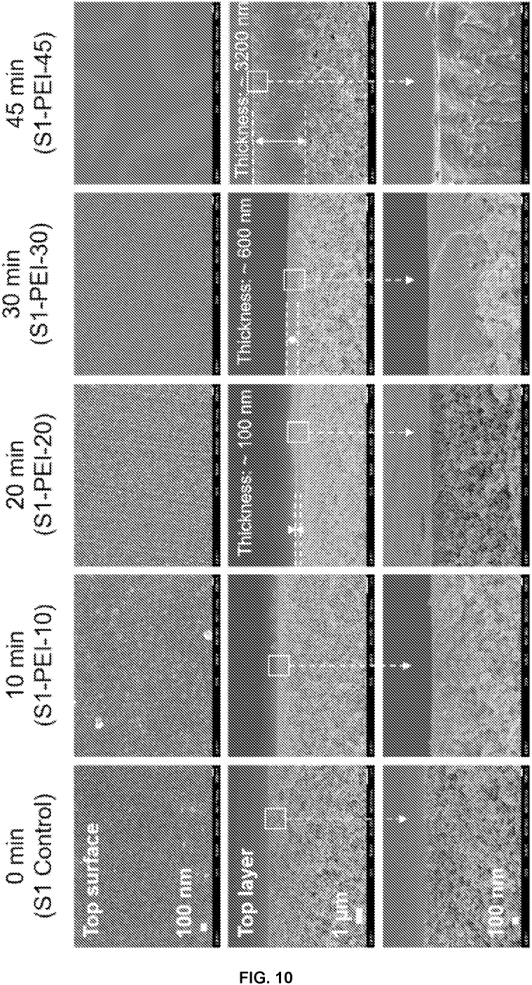

XPS was also employed to validate the degree of crosslinking in VPC/MMMs. As shown in Table 3, the N component of the skin layers in the BAPP-treated membranes rose from 5.96 % to 10.68 % when the VPC duration increased from 0 to 45 min, indicating the presence of BAPP molecules on the substrate surface. As no O element exists in these amine crosslinkers, the change of the N₁ₛ to O₁ₛ ratio (i.e. N/O) on the membrane surface may be considered as an index to the degree of crosslinking. Compared to the unmodified membrane, the N/O ratio of the 20-min VPC modified membrane only increased slightly from 0.518 to 0.580. However, the 30-min and 45-min VPC treated ones have N/O ratios almost 1- and 3-fold higher than the pristine MMM substrate, respectively. Therefore, the prolonging of the BAPP VPC duration resulted in a thicker dense-layer thickness, smaller pore size and sharper pore size distribution, but a smaller IPA permeance under a pressure of 10.0 bar and room temperature. Similar trends were observed for the APPD-treated and PEI-treated MMMs (cf. FIGS. 9-10; and Tables 1 and 4. See reaction conditions in the figures and tables).

TABLE 3

Atomic concentrations measured by XPS, pure IPA permeance and RB rejection of the pristine substrate and BAPP-modified membranes as a function of crosslinking duration. (Feed: 50 ppm RB in IPA; Pressure: 10.0 bar)

| Membrane ID | Atomic concentration (%) | | | N/O | Isopropanol (IPA) permeance (L m⁻² h⁻¹ bar⁻¹) | RH injection (%) in IPA |
|---|---|---|---|---|---|---|
| | C | O | N | | | |
| S1 (Control) | 22.54 | 11.50 | 5.95 | 0.518 | 1.90 ± 0.05 | 60.8 ± 0.56 |
| S1-BAPP-10 | 80.09 | 12.90 | 7.01 | 0.543 | 1.16 ± 0.13 | 80.6 ± 0.08 |
| S1-BAPP-20 | 80.08 | 12.61 | 7.31 | 0.580 | 1.22 ± 0.43 | 89.7 ± 0.10 |
| S1-BAPP-30 | 81.03 | 10.36 | 8.63 | 0.831 | 0.80 ± 0.33 | 99.5 ± 0.32 |
| S1-BAPP-45 | 83.96 | 5.35 | 10.68 | 1.996 | 0.30 ± 0.12 | 99.9 ± 0.01 |

TABLE 4

Atomic concentrations measured by XPS, pure IPA permeance and RB rejection of the APPD- and PEI-modified membranes as a function of crosslinking duration. (Feed: 50 ppm RB in IPA; Pressure: 10.0 bar)

| Membrane ID | Atomic concentrations (%) | | | N/O | Isopropanol permeance (L m⁻² h⁻¹ bar⁻¹) | RB rejection (%) in IPA |
|---|---|---|---|---|---|---|
| | C | O | N | | | |
| S1-APPD- 10 | 81.01 | 9.55 | 9.44 | 0.988 | 0.56 ± 0.07 | 88.9 ± 0.16 |
| S1-APPD- 20 | 80.83 | 9.30 | 9.87 | 1.061 | 0.35 ± 0.05 | 99.6 ± 0.04 |
| S1-APPD- 30 | 80.22 | 8.93 | 10.85 | 1.215 | 0.28 ± 0.01 | 99.9 ± 0.06 |
| S1-APPD- 45 | 82.83 | 6.81 | 10.34 | 1.518 | 0.15 ± 0.01 | -100% |
| S1-PEI-10 | 80.75 | 10.75 | 8.5 | 0.723 | 0.53 ± 0.02 | 95.2 ± 2.31 |
| S1-PEI-20 | 80.94 | 10.52 | 8.54 | 0.312 | 0.44 ± 0.08 | 98.7 ± 0.48 |
| S1-PEI-30 | 80.51 | 9.09 | 10.41 | 1.145 | 0.24 ± 0.11 | 99.4 ± 0.31 |
| S1-PEI-45 | 79.42 | 9.1 | 11.48 | 1.262 | No flux for 24 h | - |

FIG. 3 compares the OSN performance of all three types of VPC/MMMs made from the same VPC duration of 30 min under a pressure of 10.0 bar and room temperature. All 30 min-VPC/MMMs have RB rejections of > 99%, indicating the possession of a defect-free selective layer. However, Table 5 shows that they have significantly distinct N/O values (i.e. 1.215, 0.831 and 1.145 of APPD, BAPP and PEI, respectively). The discrepancy in N/O values may arise from the fact that the physicochemical properties of crosslinkers play an important role in determining the degree of crosslinking once the VPC duration exceeds a threshold, which is about 30 min in this study. In other words, the separation performance of VPC modified membranes is more likely to be affected by the physicochemical properties of crosslinkers rather than the VPC duration. Therefore, the effect of crosslink monomers, especially the molecular structure, on OSN performance was investigated in the next example.

TABLE 5

Atomic concentrations measured by XPS, R parameter, dense-layer thicknesses, pure IPA permeance and RB rejection of the pristine substrate and 30 min-VPC/MMMs. (Feed: 50 ppm RB in IPA; Pressure: 10.0 bar)

| Membrane ID | Atomic concentrations (%) | | | N/O | N/C | Thickness of dense layer (mm) | R parameter | Isopropranol permeance (L m⁻² h⁻¹ bar⁻¹) | RB rejection (%) in IPA |
|---|---|---|---|---|---|---|---|---|---|
| | C | O | N | | | | | | |
| S1 (Control) | 82.54 | 11.50 | 5.96 | 0.518 | 0.072 | - | 0.4963 | 1.90 ± 0.05 | 60.8 ± 0.56 |
| S1-APPD-30 | 80.22 | 8.93 | 10.85 | 1.215 | 0.135 | -1800 | 0.4646 | 0.28 ± 0.01 | 99.9 ± 0.06 |
| S1-BAPP-30 | 81.03 | 10.36 | 8.61 | 0.831 | 0.106 | -1000 | 0.4673 | 0.80 ± 0.33 | 99.5 ± 0.32 |
| S1-PEI-30 | 80.51 | 9.09 | 10.41 | 1.145 | 0.129 | -600 | 0.4565 | 0.24 ± 0.11 | 99.4 ± 0.31 |

### Example 6. Effect of Molecular Structure of Vapour-phase Crosslinkers on VPC/MMMs

The effect of the molecular structure of vapour-phase crosslinkers on VPC/MMMs (prepared in Example 3) was investigated by following the OSN performance tests described in Example 4 at 10.0 bar and room temperature unless otherwise stated.

### Results and Discussion

The major differences among these three chosen vapour-phase crosslinkers are their sizes, shapes and reactive functional groups. APPD and BAPP crosslinkers were firstly compared because they have comparable N/C atomic ratios (i.e. 4/9 and 4/10). As tabulated in Table 5, S1-APPD-30 has a higher N atomic concentration, N/O and N/C atomic ratios than S1-BAPP-30, suggesting the former has a higher degree of crosslinking than the latter. Since APPD and BAPP have the same number of —NH₂ groups available to react with imide chains of Matrimid®, their discrepancy in degree of crosslinking degree may arise from different molecular sizes and geometries (A. O. Aleshinloye, J. B. Bodapati & H. Icil, J. Photochem. Photobiol. A: Chem. 2015, 300, 27-37; and A. Mehta & A. L. Zydney, J. Membr. Sci. 2008, 313, 304-314). Although APPD has a longer molecular length than BAPP (FIG. 1), the latter has a bigger molecular volume than the former (183 vs. 104 Å3). Consequently, the small and elongated APPD molecules can diffuse faster than the large BAPP ones (D. Yoo et al., Phys. Chem. Chem. Phys. 2019, 21, 1484-1490). A comparison of their density-layer thickness as a function of VPC duration at 120° C. and 1 atm (FIGS. 4 and 9) confirmed our hypothesis. The small and slender APPD molecules induced a thicker crosslinked dense-selective layer than the large and fat BAPP ones under the same VPC duration. For example, the APPD-modified MMMs have dense layer thicknesses of ~300, 1800 and 2900 nm for VPC durations of 20, 30 and 45 min, respectively. In contrast, the corresponding thicknesses of BAPP-modified ones are ~200, 1000 and 2700 nm, respectively.

Table 5 compares the dense layer properties and OSN performance of all 30-min VPC modified MMMs under a pressure of 10.0 bar and room temperature. The bulky PEI molecules have a MW of 800 g mol⁻¹, molar volume of 1806 Å³, and low vapour diffusion rate. As a result, the S1-PEI-30 sample has the thinnest selective layer of ~600 nm as compared to S1-APPD-30 of ~1800 nm and S1-BAPP-30 of ~1000 nm. Surprisingly, even though it has the thinnest selective layer, it had the lowest IPA permeance among these three VPC modified membranes (i.e. 0.24 vs. 0.28 and 0.8 L m⁻² h⁻¹ bar⁻¹). Clearly, its hyper-branch characteristics and multiple amine function groups resulted in a more densified layer with a smaller free volume.

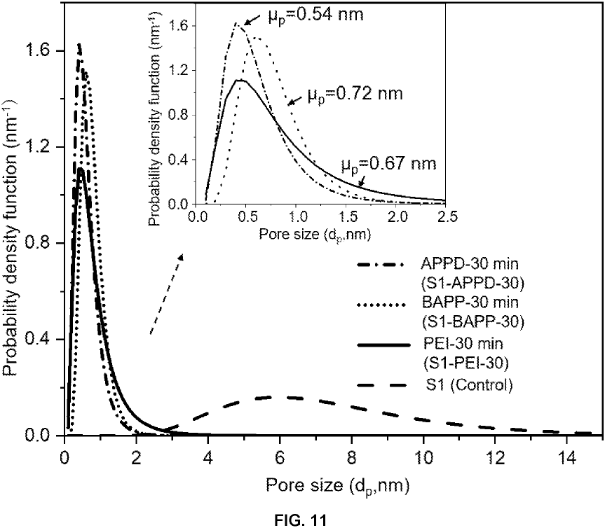

The R parameter, pore size and pore size distribution of 30-min VPC modified membranes shown in Table 5 and FIG. 11 provided convincing information in line with the above hypothesis. The PEI-modified membrane has the smallest R value, implying that it has the lowest free volume in the dense layer. In addition, the APPD-modified membrane has a more compacted dense layer than the BAPP-modified one. Interestingly, their mean effective pore sizes (µₚ) follow the order of BAPP- > PEI- > APPD-modified membranes (i.e. 0.72, 0.67 and 0.54, respectively). This trend suggests that APPD and PEI vapour are more effective in generating smaller pores in the dense-selective layer than the BAPP vapour. Besides, the S1-APPD-30 membrane has the narrowest pore size distribution, probably owing to the linear molecular shape of the APPD crosslinker. In other words, the slender APPD can diffuse deeper and pack tightly together while the irregularly-shaped BAPP containing a nitrogen-containing six-membered ring prevent itself from packing tightly (B. T. Low et al., Macromolecules 2008, 41, 1297-1309).

Although both PEI and BAPP molecules contain tertiary amine groups and non-linear structures, the former has more reactive amine groups than the latter. The highly bulky and branched PEI with six reactive amine groups per unit monomer may facilitate the crosslinking reaction everywhere and form a network with great tortuosity (C. Li et al., Prog. Org. Coat. 2019, 132, 429-439; and J. J. Virgen-Ortíz et al., J. Mater. Chem. B 2017, 5, 7461-7490). Therefore, S1-PEI-30 has a higher N/O ratio (i.e. the degree of crosslinking) than S1-BAPP-30 (i.e. 1.145 vs. 0.831), as shown in Table 5. The R parameter also confirms this trend because the former has a lower R parameter (i.e. a tighter structure) than the latter (i.e. 0.4565 vs. 0.4673). However, a comparison of their pore size distributions (FIG. 11) and IPA permeances (Table 5) indicates that both have comparable mean pore sizes (i.e. 0.67 vs. 072 nm), but S1-PEI-30 has a broader pore size distribution and a surprisingly lower IPA permeance than S1-BAPP-30. These phenomena may arise from the fact that the highly branched and reactive amino groups of PEI may form a dense-selective layer with a tortuous network full of steric hindrance for solvent transport (A. Mehta & A. L. Zydney, J. Membr. Sci. 2008, 313, 304-314).

As a consequence, one may derive the following conclusions from the above findings: (1) the molecular size and shape of crosslinkers are the dominant factors in determining the pore size of VPC modified membranes if the crosslinkers have the same number of reactive functional groups; and (2) the number of reactive functional groups becomes the primary factor in determining the pore size if the crosslinkers have a non-linear irregular molecular geometry.

### Example 7. Characterization of MMM/PET Non-woven Substrates

To take advantages of the extraordinary robustness of PET non-woven fabrics, VPC/MMMs were also prepared on PET fabrics in Example 3 to mimic industrial situations for OSN applications.

### Results and Discussion

Polyimide polymers are commonly reinforced with PET non-woven fabrics for the formation of stable OSN membranes (S. P. Sun et al., AIChE J. 2014, 60, 3623-3633; and US 20120223014 A1). The adverse influences of non-woven fabrics on OSN performance may be minimized due to their large porosity. Although the resultant MMM/PET substrates have a thick entire thickness, the thickness of the asymmetric MMMs declines dramatically because some of the mixed matric materials are squeezed downward to the porous PET supports during the casting.

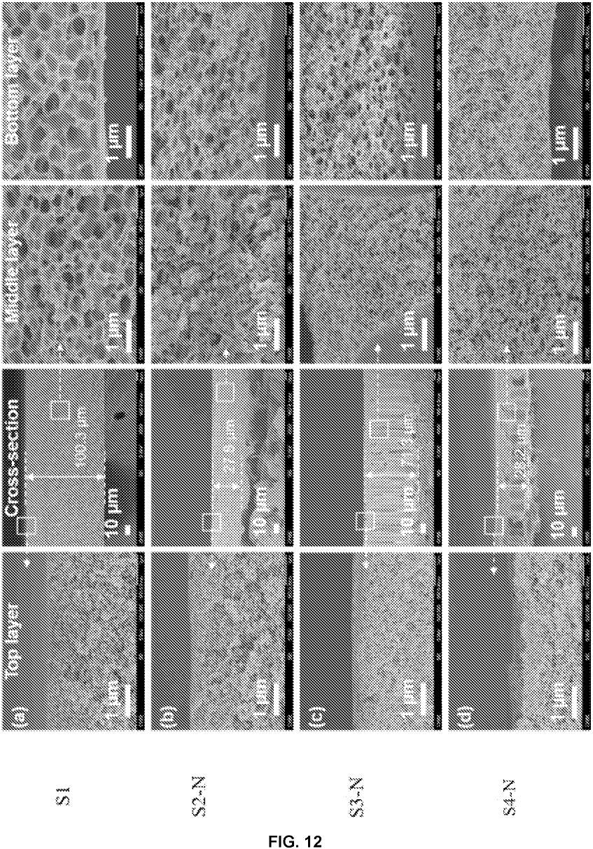

As a result, under identical casting conditions (pressure = 10.0 or 5.0 bar), the thickness of the MMMs on PET fabrics declined by 2-fold from 100.3 ± 2.7 µm to 27.8 ± 3.2 µm (i.e. S1 vs. S2-N in FIG. 12). This led to a significant increase in the solvent permeance. As displayed in the first two rows of Table 6, the MMM substrate cast on PET fabrics has a pure IPA permeance 1.5-fold higher than that without fabrics (i.e. 4.74 vs. 1.90 L m⁻² h⁻¹ bar⁻¹ in Table 6).

TABLE 6

Pure IPA permeance and RB rejection of the pristine substrate and substrates consisting of PET non-woven fabrics^{a}. (Feed: 50 ppm RB in IPA; Pressure: 10.0 or 5.0 bar)

| Membrane ID | Polyester non- woven | Testing set- up | Pressure (Bar) | Pure Isopropanol permeance (Lm⁻²h⁻ ¹bar⁻¹) | Isopropanol permeance (Lm⁻²h⁻ ¹bar⁻¹) | RB rejection (%) in IPA |
|---|---|---|---|---|---|---|
| S1 | No | Dead-end | 10 | 1.90 ± 0.05 | 1.18 ± 0.02 | 60.8 ± 0.56 |
| S2-N | Yes | Dead-end | 10 | 4.74 ± 0.04 | 2.32 ± 0.43 | 63.9 ± 1.62 |
| S3-N | Yes | Cross-flow | 5 | 4.98 ± 0.06 | 4.18 ± 0.01 | 78.3 ± 1.16 |
| S4-N | Yes | Cross-flow | 5 | 20.53 ± 1.82 | 14.35 ± 0.84 | 56.04 ± 0.51 |

^{a} The preparation conditions of S1, S2-N, S3-N and S4-N are shown in Table 2.

The morphology and performance of the MMM/PET substrates are dependent on (1) the solvent chemistry in the coagulant and crosslinking bath; and (2) the gap of the casting knife during phase inversion. The replacement of IPA with DI water in the phase inversion and diamine crosslinking bath resulted in a larger number of finger-like macrovoids in the cross-section (FIGS. 12c-d, Z. F. Gao et al., J. Membr. Sci. 2019, 574, 124-135). A further reduction in the knife gap to 100 µm gave a resultant substrate (S4-N) that has a top MMM layer of 28.2 µm thick which is almost as thin as the S2-N substrate of 27.8 µm. Interestingly, the former had a 6-fold higher IPA permeance than the latter (i.e. 14.35 vs. 2.32 L m⁻² h⁻¹ bar⁻¹ in Table 6) without significantly compromising the RB rejection (i.e. 56.04 vs. 63.9%). The flux enhancement and high rejection may result from the existence of macrovoids in the cross-section to lower transport resistance and the formation of a tight skin surface owing to the fast demixing between NMP and water, respectively (C. A. Smolders et al., J. Membr. Sci. 1992, 73, 259-275).

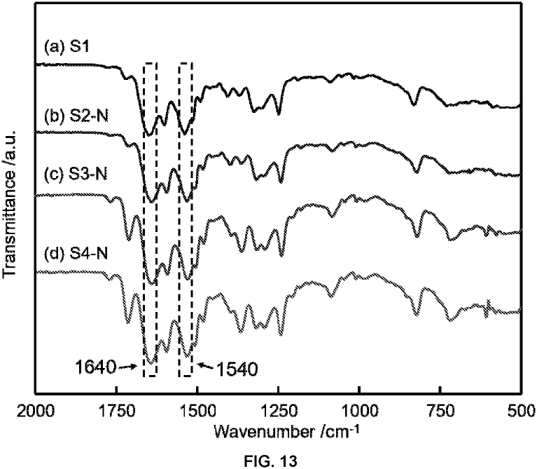

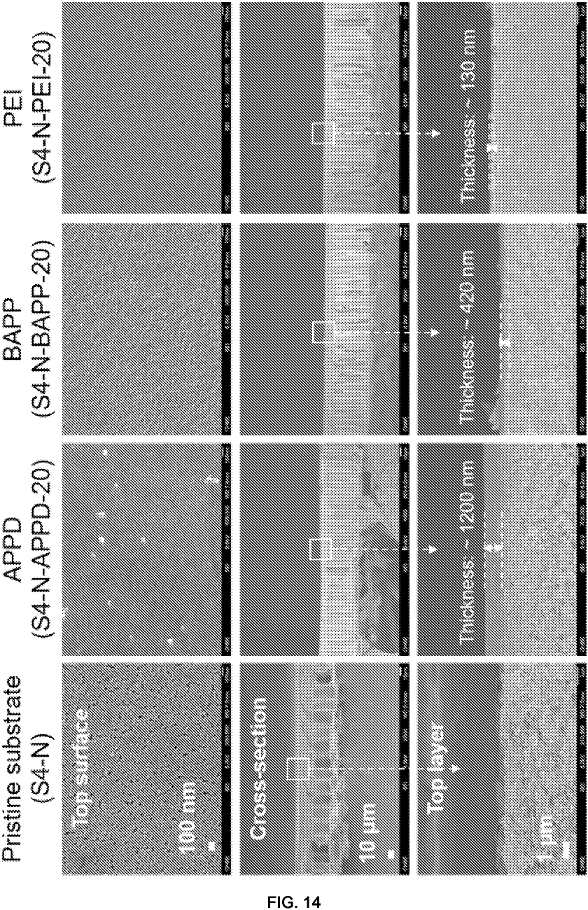

FIG. 13 shows the FTIR spectra of the HDA-crosslinked MMM/PET substrates. All the HDA-crosslinked MMM/PET substrates have clear amide peaks at 1540 cm⁻¹ and 1640 cm⁻¹, inferring the success of HDA crosslinking modification (Z. F. Gao et al., J. Membr. Sci. 2018, 565, 169-178; and S. P. Sun et al., AIChE J. 2014, 60, 3623-3633). After being immersed in MeOH, EtOH, IPA, DMF, THF, toluene and n-hexane for 14 days, none of them show delamination. Since the S4-N substrate has the highest IPA permeance (Table 6) and uses water instead of IPA in the coagulation and crosslinking bath, its manufacture process is greener and more economical than others. Therefore, it was selected to form a series of VPC/MMM/PET membranes modified by different amine vapour for 20 min for the subsequent studies. FIG. 14 shows their morphologies and dense-layer thicknesses.

### Example 8. OSN Performance of VPC/MMM/PET Membranes

The VPC/MMM/PET membranes prepared in Example 3 were taken for OSN performance tests in IPA as described in Example 4, under a pressure of 10.0 bar and room temperature.

### Results and Discussion

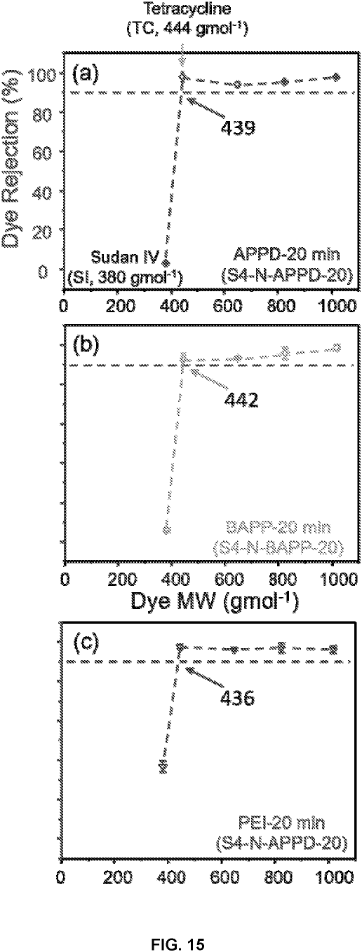

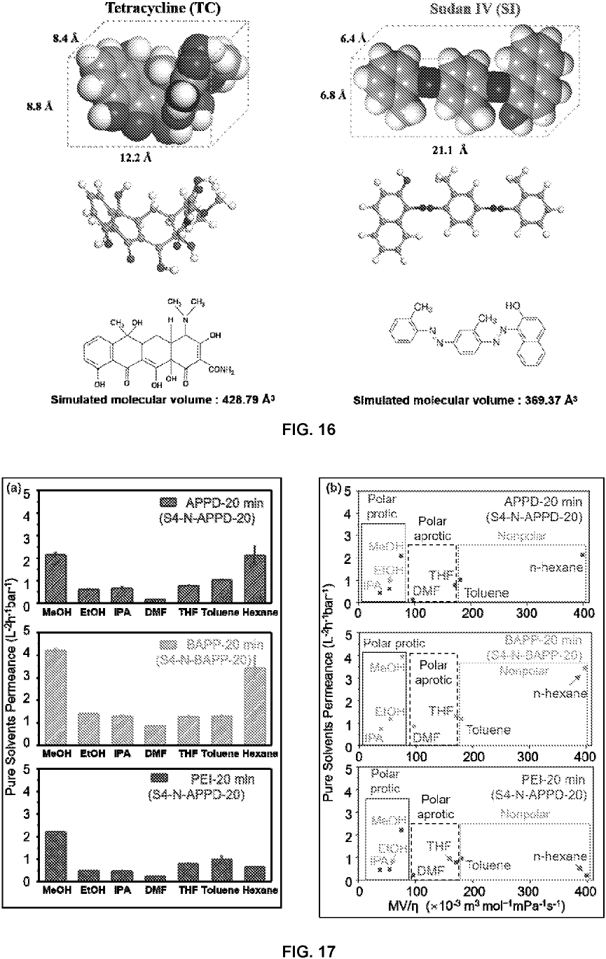

FIG. 15 shows that the three types of VPC/MMM/PET hybrid membranes have MWCO values of 439, 442 and 436 g mol⁻¹, respectively. Interestingly, all the membranes displayed a steep relationship between rejection and MW near the MWCO region. For example, the APPD-modified VPC/MMM/PET membrane (S4-N-APPD-20) has a much lower rejection of SI than TC (i.e. 3.12 vs. 97.83 %) even though their MW values are very close (i.e. 380 vs. 444 g mol⁻ ¹). Similar phenomena were observed for the BAPP- and PEI-modified VPC/MMM/PET membranes (i.e. S4-N-BAPP-20 and S4-N-PEI-20). As both SI and TC are neutral molecules, the charging effect on the solute rejection is negligible (Z. F. Gao et al., J. Membr. Sci. 2019, 574, 124-135; D. F. Stamatialis et al., J. Membr. Sci. 2006, 279, 424-433; and X. Li et al., Chem. Mater. 2008, 20, 3876-3883). The distinct SI and TC rejections may be caused by their discrepancies in molecule size and shape. SI and TC have similar molecular volumes of 369.37 Å3 and 428.79 Å3 (details of 3D molecular structures are shown in FIG. 16), respectively. However, the former displays a planar molecular structure with a 6.8 Å × 6.4 Å cross-section area, while the latter possesses a triangular pyramidal shape with a bigger dimension of 8.8 Å × 8.4 Å than the former. Consequently, the triangular pyramidal structure and the slightly large cross-section area of TC molecules are rejected by VPC densified skin layers. Comparing with commercially available OSN membranes, these VPC modified membranes have much smaller MWCO values (G. M. Shi et al., J. Membr. Sci. 2019, 588, 117202). Thus, they may have special potential to concentrate or separate small pharmaceutical intermediates.

Therefore, a short exposure duration (i.e. no more than 30 min) of HDA-crosslinked mixed matrix substrates with amine vapour can create a defect-free ultrathin dense skin layer with a sharp and narrow pore size distribution. It converted the pore size of HDA crosslinked mixed matrix substrates from an ultrafiltration (UF) to nanofiltration (NF) range.

### Example 9. Transport Behaviour Across VPC/MMM/PET Membranes

Six more organic solvents (MeOH, EtOH, THF, DMF, n-hexane and toluene) were further chosen to examine transport behaviour across these VPC/MMM/PET composite membranes (prepared in Example 3) by following the OSN performance tests described in Example 4, under a pressure of 5.0 bar and room temperature.

### Results and Discussion

As shown in FIG. 17, the permeances of all the membranes generally obey the following trends: (i) for polar protic solvents, MeOH> EtOH ≥ IPA; (ii) for polar aprotic solvents, THF > DMF; and (iii) for nonpolar solvents: n-hexane > toluene. Table 7 summarizes some physical characteristics of these solvents. Regardless of the types of solvents (polar, nonpolar or aprotic), almost all the permeances declined with a decrease in MV η⁻¹, a combined term from molar volume (MV) and viscosity (η) (X. Li et al., Chem. Mater. 2008, 20, 3876-3883; and S. Veríssimo, K. V. Peinemann & J. Bordado, J. Membr. Sci. 2006, 279, 266-275). However, the PEI-modified membrane (S4-N-PEI-20) has a peculiar order for n-hexane and toluene permeances. Why does toluene have a higher permeance of 0.98 L m⁻² h⁻¹ bar⁻¹ than n-hexane of 0.64 L m⁻² h⁻¹ bar⁻¹? The former has a smaller MV than the latter but its 2D geometry and large kinetic diameter (5.9 Å) usually results in a lower permeance than 1-D n-hexane with a small kinetic diameter (4.3 Å) (H. Wu et al., Chem. Rev. 2012, 112, 836-868; and W. Wei, J. Liu & J. Jiang, ACS Sustainable Chem. Eng. 2019, 7, 1734-1744). Thus, the interesting reverse order of permeance may be due to the influences of solvent-membrane interactions (Y. Ji et al., ACS Appl. Mater. Interfaces 2019, 11, 36717-36726). Firstly, n-hexane has an extremely nonpolar property of 0.009 relative polarity (P), while toluene has a higher P of 0.099.

As a result, the PEI-modified membrane containing abundant —NH₂ groups should have better interactions with toluene than n-hexane (S. Singh et al., J. Membr. Sci. 1998, 142, 111-127; and C. Li et al., Prog. Org. Coat. 2019, 132, 429-439). Secondly, the contact angles shown in Table 8 support our hypothesis. The PEI modified membrane (S4-N-PEI-20) has a much lower contact angle of toluene than that of n-hexane (17.27° vs. 63.17°), indicating that the former has a stronger interaction with the membrane surface than the latter (Y. Ji et al., ACS Appl. Mater. Interfaces 2019, 11, 36717-36726). This may lead toluene to have a higher permeance than n-hexane.

TABLE 7

Physical properties of different solvents (H. Wu et al., Chem. Rev. 2012, 112, 836-868; P. J. Linstrom & W. G. Mallard, J. Chem. Eng. Data 2001, 46, 1059-1063; and K. Hendrix et al., J. Membr. Sci. 2014, 452, 241-252)

| Solvent | MW e (g mol⁻¹) | d_{K}^{b)} (nm) | MV e (-10⁻¹ m² mol⁻¹) | η^{d)} (mPa·s) | Γ^{e)} (mNm⁻¹) (MP | δ^{f)} a^{½}) | P^{g)} | MV/η (×10⁻³ m² mol⁻¹mPa⁻¹s⁻¹) |
|---|---|---|---|---|---|---|---|---|
| MeOH | 32.04 | 0.38 | 40.7 | 0.54 | 22.7 | 29.6 | 0.762 | 75.37 |
| NaOH | 46.0 | 0.43 | 58.8 | 1.08 | 22.1 | 26.5 | 0.654 | 54.44 |
| IPA | 60.1 | 0.47 | 76.8 | 2.05 | 23.0 | 23.6 | 0.546 | 37.46 |
| DMF | 73.1 | 0.55 | 77.1 | 0.80 | 37.1 | 24.9 | 0.386 | 96.25 |
| THF | 72.1 | 0.49 | 81.2 | 0.48 | 26.4 | 19.4 | 0.207 | 169.17 |
| Tuluene | 92.14 | 0.59 | 106.3 | 0.59 | 28.4 | 18.2 | 0.099 | 180.17 |
| n-hexane | 86.2 | 0.43 | 131.4 | 0.33 | 18.43 | 14.9 | 0.009 | 398.18 |

^{*} ^{a)} MW is the solvent molecular weight; ^{b)} d_{K} is the solvent kinetic diameter; ^{c)} MV is the solvent molar volume; ^{d)} η is the solvent dynamic viscosity; ^{e)} Γ is the solvent surface tension; ^{f)} δₚ is the Hansen solubility parameter of the solvent polarity; and ^{g)} P is the relative solvent polarity.

TABLE 8

Solvents permeance and contact angles of various solvents on VPC/MMM/PET composite membranes

| Solvent | Pure solvents permeace (Lm⁻²h⁻¹bar⁻¹) at 5 bar | | | | | |
|---|---|---|---|---|---|---|
| | APFD-20 min² | Solvent contact angle (°) | BAPF-20 min² | Solvent contact angle (°) | PEI-20 min | Solvent contact angle (°) |
| MeOH | 2.14 ± 0.09 | 9.09 ± 0.78 | 4.18 ± 0.09 | 7.62 ± 0.24 | 2.22 ± 0.01 | 26.93 ± 3.87 |
| BtOH | 0.61 ± 0.03 | 6.84 ± 0.90 | 1.39 ± 0.03 | 6.78 ± 0.53 | 0.48 ± 0.01 | 18.74 ± 2.51 |
| IPA | 0.66 ± 0.05 | 0.00 | 1.28 ± 0.04 | 0.00 | 0.47 ± 0.02 | 11.07 ± 2.73 |
| DMF | 0.15 ± 0.01 | 38.30 ± 4.23 | 0.86 ± 0.01 | 24.33 ± 2.67 | 0.23 ± 0.01 | 29.41 ± 0.59 |
| THF | 0.77 ± 0.02 | 11.00 ± 0.44 | 1.25 ± 0.04 | 9.63 ± 1.11 | 0.80 ± 0.01 | 15.77 ± 1.19 |
| Toluene | 1.03 ± 0.01 | 12.37 ± 1.01 | 1.28 ± 0.03 | 9.75 ± 2.16 | 0.98 ± 0.15 | 17.27 ± 0.93 |
| n-hexane | 2.13 ± 0.39 | 49.36 ± 6.23 | 3.44 ± 0.51 | 40.33 ± 2.13 | 0.64 ± 0.01 | 63.17 ± 5.33 |

^{a} Sample S4-N-APPD-20. ^{b} Sample S4-N-BAPP-20. ^{c} Sample S4-N-PEI-20.

In addition, compared to APPD- and BAPP-modified membranes, Table 8 shows that the PEI-modified membrane has the highest contact angles of n-hexane and toluene, implying that it has the lowest affinity with non-polar solvents. Consequently, it has the lowest n-hexane and toluene permeances among these three membranes. The PEI-modified membrane may be therefore more suitable for OSN applications involving polar and aprotic solvents, while APPD-and BAPP-modified membranes are for applications involving wider solvents.

### Example 10. 120-h OSN Performance of VPC/MMM/PET Membranes

The VPC/MMM/PET membranes prepared in Example 3 were taken for OSN performance tests as described in Example 4 at 5.0 bar except for 120 h. Four solvent systems were prepared and each system contained 50 ppm of TC (MW =444 g mol⁻¹).

### Results and Discussion

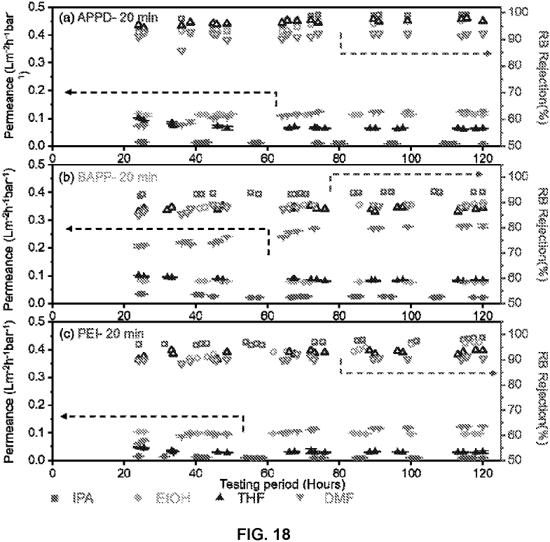

FIG. 18 plots the permeance and rejection as a function of time in 120-h continues cross-flow tests of the newly developed VPC/MMM/PET membranes under 5.0 bar. All three types of VPC membranes displayed stable performances in terms of permeance and rejection, indicating (i) no evidence of severe membrane compaction under 5.0 bar; and (ii) negligible TC fouling in various solvent feeds within 5 days. Consistent with our previous conclusion in Example 6 that APPD and PEI vapour are more effective in generating smaller pores in the dense-selective layer than the BAPP vapour, FIG. 18 reconfirms the same observation. The VPC/MMM/PET membranes modified by the former two crosslinkers exhibited higher rejections than that modified by the latter. Interestingly, the rejection of TC also varied slightly in these 4 solvents with an order of IPA > EtOH ≥ THF > DMF. The hybrid VPC/MMM cast on PET non-woven fabrics showed more than 90% rejection rates of TC in four selected organic solvents (i.e. EtOH, IPA, THF and DMF) over 120 h under continuous cross-flow filtration tests without any remarkable fluctuation. This is due to different solvent interactions with the membranes. Usually, crosslinked polyimides may be slightly swollen by DMF (P. Marchetti et al., Chem. Rev. 2014, 114, 10735-10806), thus the TC-DMF has the lowest rejection. Future studies will be conducted to overcome it.

### Comparative Example 1

The solvent permeance of the VPC modified membranes prepared in Example 3 was compared to commercial membranes with similar MWCO values.

### Results and Discussion

Table 9 compares the solvent permeance of the VPC modified membranes in this work with other composite OSN membranes with similar MWCO values. The higher permeances achievable for the BAPP/MMM/PET membranes offer a clear advantage over pure polymeric or one-step mixed matrix OSN membranes. Meanwhile, there is no hexane permeance for DuraMem and PuraMem membranes. The wider range of solvents applicable for our newly developed membranes show great potential of VPC membranes for commercialization to tackle industrial demands. Furthermore, since the proposed VPC procedure uses a small amount of amine vapour and there is no much waste production, the post-polymerization VPC modification via amine vapour may offer a greener, more environmentally-friendly and scalable process to fabricate the next generation of OSN membranes.

TABLE 9

A comparison of solvent permeance between VPC/MMM/PET hybrid membranes and other literature and commercial membranes with similar MWCO values

| Membrane | Pure solvents permeasure | (I.co *t.₄-. ) | MWCO (g ⁻¹) in wxcseon solvents | Ref. |
|---|---|---|---|---|
| NM2MWCNTs/P34 | BtOH | 2.3 | - | M. H. D. A. Farabaoi et al., Chem. Eng. J. 2018, 3?5. 174-185 |
| | IPA | 0.8 | 437 | |
| sPP5tl/PEI | EtOH | 10 | 500 | Y. Feng et al., J. Membr. Sci. 2018, 549, 550-558 |
| sPP5tl/PEI/GA | EtOH | 1.4 | 300 | |
| PBEK | THF | 0.32 | 400 | I. do Silva Burgal et al., J. Membr Sci. 2015, 479, 105-116 |
| | DMP | 0.07 | 470 | |
| Doument 500 | MeOH | 1.46 | 500* | Cr M. Shi et al., J. Membr. Sci. 2019, 588, 117202 |
| | EtOH | 0.82 | | |
| | DMP | 2.45 | | |
| | Hexone | 0 | | |
| Dazavyrin 300 | MeOH | 0.92 | 500* | |
| | EtOH | 0.27 | | |
| | DMF | 0.39 | | |
| | Hexane | 0 | | |
| Prxerrmr 280 | MeOH | 3.28 | 230* | |
| | EtOH | 0.65 | | |
| | Hexane | 0 | | |
| S4-MBAPP-20 | MeOH | 4.16 | - | T is work |
| | EtOB | 1.39 | - | |
| | IFA | 1.36 | 442 | |
| | DMP | 0.86 | - | |
| | THF | 1.25 | - | |
| | o-hexane | 3.44 | - | |

^{a} The MWCO data were obtained from the supplier of Evonik commercial OSN membranes.

### Example 11. Fabrication of Crosslinked PBI Hollow Fiber

The polymer dope of PBI/DMAc/LiCl/propanol/PVP (17.5/60.5/1.5/17.5/3.0 wt%) was prepared. The original PBI dope with additives of LiCl and PVP was diluted by DMAc and propanol as the solvent and co-solvent, respectively, and stirred overnight at 50° C. until a homogenous solution was obtained. The dope solution was then allowed to stand still and degas for one day. Next, the solution was loaded into a 500 mL ISCO syringe pump and further degassed overnight prior to spinning. A mixture of DMAc/IPA (15/85 wt%) was adopted as the bore fluid. The hollow fibers were spun using a dry-jet wet-spinning technique where the dope solution was fed into the outer annulus of the spinneret while the bore fluid was fed into the inner annulus (FIG. 19). Both extruded streams were allowed to pass through a 2.0 cm air gap before entering an IPA/water (50/50 wt%) coagulation bath. The hollow fibers were then collected on a take-up drum. The dope composition, spinning conditions and post treatments were listed in Table 10. The nascent hollow fibers were soaked in DI water for 48 h to remove residual solvents and chemicals. Subsequently, as-spun hollow fibers were immersed in tap water for 48 h to remove residual solvents and chemicals. Afterward, hollow fibers were immersed in a 5/95 wt% DBX/IPA solution with stirring at 80° C. for 24 h and then rinsed by IPA to remove the remaining chemicals. Next, the hollow fibers were immersed in a mixture of IPA/glycerol (50/50 wt%) for 48 h and dried in air under ambient conditions.

TABLE 10

Summary of dope compositions, spinning conditions and post-treatments of polyimide hollow fiber supports

| | |
|---|---|
| Dope solution composition (wt %) | 17.5/60.5/1.5/17.5/3.0 (PBI/DMAc/LiCI/propanol/PVP K15) |
| Bore fluid solution composition (wt %) | 15/85 (DMAc/IPA) |
| Dope flow rate (mL/min) | 3.2 |
| Bore fluid flow rate (mL/min) | 1.0 |
| Air-gap length (cm) | 2.0 |
| Take-up speed (m/min) | 6.2 |
| External coagulants | IPA-Water |
| Spinneret dimension (mm) | Spinneret 1.2-0.5 |
| Spinning temperature (°C) | Ambient (23° C.) |

### Example 12. Synthesis of Continuous UiO-66-NH₂ Membranes and Vapor-Phase EDA Modification

### PBI-UiO66-NH₂

The ends of the crosslinked PBI hollow fibers prepared in Example 11 were sealed with epoxy. The prepared hollow fibers were first immersed in ACD ligand solution overnight prior to the introduction of ZrCl₄ solution at room temperature. The in situ solvothermal synthesis of UiO-66-NH₂ was carried out at 120° C. for 48 h. The procedure is detailed below:

Step 1. The ligand solution was prepared by dissolving ACD (0.186 g or 1.03 mmol) in 8 mL of DMF/AA mixed solvent (volume ratio of 3:1) and stirred in a glass vial at room temperature. Subsequently, the crosslinked PBI membrane was pre-treated by soaking in the as-prepared solution at room temperature for 12 h.

Step 2. ZrCl₄ (0.08 g or 0.34 mmol) was dissolved in a mixture of 12 mL of DMF/AA (volume ratio of 1:1) and DI water (0.075 µL) under stirring and heated to 120° C. for 12 h.

Step 3. The Zr solution from step 2 was added to the membrane/ligand solution system from step 1. The in situ solvothermal synthesis of MOF layer took place at 120° C. for 48 h during which a noticeable change in the solution color from yellow to creamy was observed. The resultant membrane was then immersed in ethanol solution to remove residuals and stored in ethanol solution for further use.

### TAEA-PBI-UiO66-NH₂

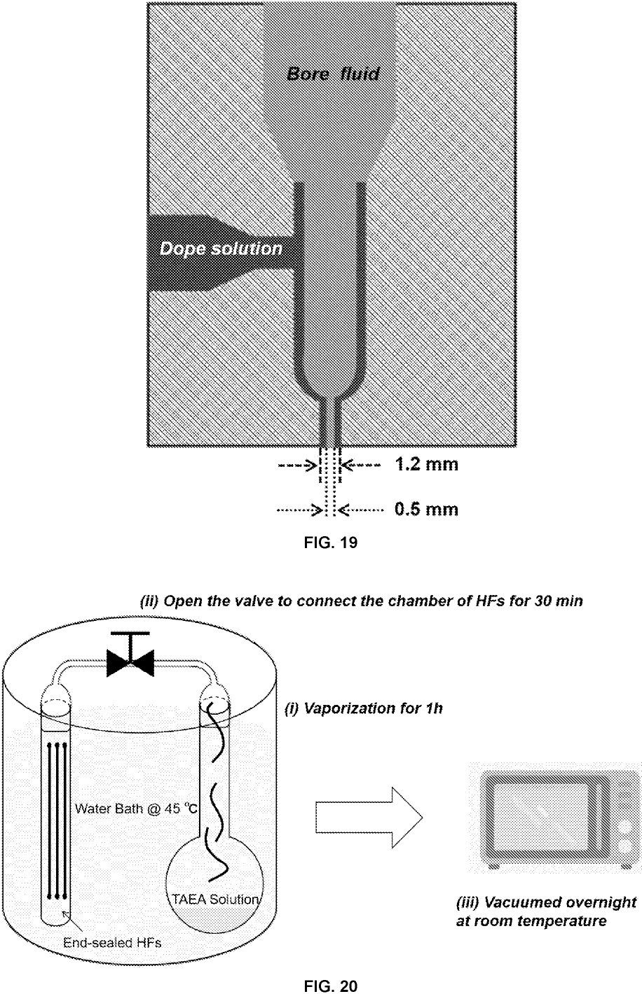

TAEA-PBI-UiO66-NH₂ was prepared from PBI-UiO66-NH₂ via vapor-phase EDA modification. The set-up of vapor crosslinking is shown in FIG. 20. 100 mL of TAEA in a 45° C. water bath was allowed to stabilize over 1 h. Upon stabilization, the epoxy sealed fibers were quickly exposed to the vapor phase TAEA for 30 min. After stipulated vapor phase modification times, the surface-modified fibers were removed from the containment and placed in a vacuum environment overnight at room temperature.

### Example 13. OSN Hollow Fiber Membranes (HFMs) Performance Tests

PBI-UiO66-NH₂ and TAEA-PBI-UiO66-NH₂ prepared in Example 12 were taken for OSN performance tests as described below.

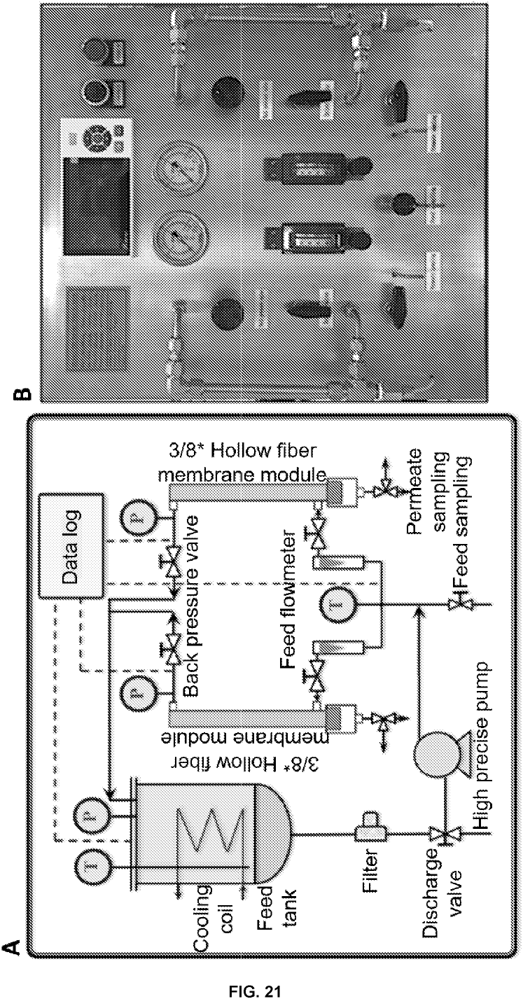

The OSN performance of hollow fiber including pure solvent permeance (L m⁻² h⁻¹ bar⁻¹) and their dye rejection in ethanol were measured using a solvent-resistant stainless steel crossflow setup (FIG. 21) operated at 1 bar and room temperature. The permeance (L m⁻² h⁻¹ bar⁻¹) was calculated using Eq. (6) and (7). The solute concentration in each organic solution during the rejection tests was fixed at 50 ppm. The solute rejection was calculated using Eq. (2). The dye concentration in different organic solvents was measured by a UV-Vis spectrometer (Pharo 300, Merck). The MWCO was determined when R = 90%.

### Results and Discussion

The OSN performance of PBI-UiO66-NH₂ and TAEA-PBI-UiO66-NH₂ is shown in Table 11.

TABLE 11

OSN performance of HFMs. (Feed: 50 ppm BBR in EtOH; Pressure: 5.0 bar)

| Membrane | Solvents permeance (Lm⁻²h⁻¹bar⁻¹) | | Solute | MW of the solute (gmol⁻¹) | Rejection (%) |
|---|---|---|---|---|---|
| PBI-UiO66-NH₂ | EtOH | 1.89 | Brilliant Blue R | 826 | 86.13 |
| TAEA-PBI-UiO66-NH₂ | EtOH | 1.12 | Brilliant Blue R | 826 | 97.16 |

1. An organic solvent nanofiltration (OSN) membrane and/or an organic solvent reverse osmosis (OSRO) membrane comprising a composite mixed matrix material that comprises:
a solvent-resistant polymeric matrix with functional groups suitable for crosslinking; and
an inorganic material homogeneously dispersed throughout the polymeric matrix, where the mixed matrix material has a first region comprising a surface of the membrane and a second region, wherein:
the first region of the mixed matrix material further comprises a crosslinking agent that crosslinks the polymeric matrix in said first region via the functional groups suitable for crosslinking; and
the first region has a mean effective pore size of from 0.2 to 2 nm.

2. The membrane according to claim 1, wherein the solvent-resistant polymeric matrix with functional groups suitable for crosslinking is a polymeric matrix formed from one or more of the group selected from a polybenzimidazole (PBI), a poly(ether ether ketone) (PEEK), a polyacrylonitrile (PAN), and a polyimide (PI), optionally wherein the solvent-resistant polymeric matrix with functional groups suitable for crosslinking is a polymeric matrix formed from one or more of the group selected from a polybenzimidazole (PBI) or a polyimide (PI).

3. The membrane according to claim 2, wherein the polymeric matrix is formed by a polyimide.

4. The membrane according to claim 3, wherein the polyimide has a repeating unit of formula I: 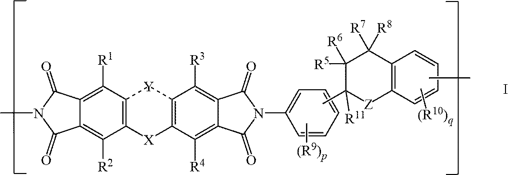
each of R¹ to R⁸ and R¹¹ is independently selected from H, C₁₋₃ alkyl, C₁₋₃ haloalkyl and halo;
X is a bond or is —C(═O)—;
Y is absent or is —C(═O)—; or
X and Y are each absent so as to form a fused aromatic structure of formula (Ia): 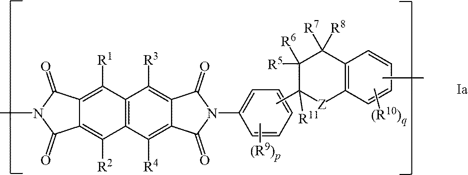
each R⁹, when present, is independently selected from the group comprising: C₁₋₃ alkyl, C₁₋₃ haloalkyl and halo;
each R¹⁰, when present, is independently selected from the group comprising: C₁₋₃ alkyl, C₁₋₃ haloalkyl and halo;
Z is selected from the group comprising: a bond and —C(R¹)₂—;
p is from 0 to 4; and
q is from 0 to 3.

5. The membrane according to claim 4, wherein the polyimide has a repeating unit of formula II: 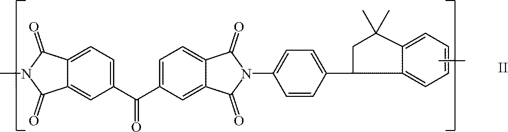
.

6. The membrane according to claim 1, wherein the inorganic material is selected from one or more of a metal organic framework (MOF), carbon nanotubes, zeolites, titanium dioxide (TiO₂), nanoalumina, silica nanoparticles, silver nanoparticles, and a graphene oxide.

7. The membrane according to claim 6, wherein the inorganic material is a MOF.

8. (canceled)

9. (canceled)

10. The membrane according to claim 1, wherein the weight to weight ratio of the polyimide polymeric matrix material to the inorganic material is from 100:1 to 1000:1, such as from 100:1 to 25:1, such as from 150:1 to 200:1, such as 180:1.

11. The membrane according to claim 1, wherein the polymeric matrix material is partially crosslinked by a diamine (e.g. 1,6-hexanediamine), optionally wherein the polymeric matrix material is a polyimide polymeric matrix material is partially crosslinked by a diamine (e.g. 1,6-hexanediamine).

12. The membrane according to claim 1, wherein the crosslinking agent is selected from one or more of the group consisting of a polyamine, and a hydrazine.

13. (canceled)

14. (canceled)

15. (canceled)

16. The membrane according to claim 1, wherein the first region has a mean effective pore size of from 0.4 to 1.2 nm, such as from 0.5 to 1.13 nm, such as 0.6 to 0.9 nm.

17. The membrane according to claim 1, wherein the mixed matrix material has a thickness of from 20 to 150 µm, such as from 25 to 110 µm.

18. The membrane according to claim 1, wherein the first region has a thickness of from 100 to 5,000 nm, such as from 200 to 3,500 nm, such as from 300 to 3,000 nm, such as from 500 to 2,500 nm.

19. (canceled)

20. (canceled)

21. (canceled)

22. The membrane according to claim 1,
wherein the membrane displays a molecular weight cut-off value of from 100 to 500 Daltons, optionally wherein:
(a) when the membrane is an OSN membrane it displays a molecular weight cut-off value of from 400 to 500 Daltons or when the membrane is an OSRO membrane it displays a molecular weight cut-off value of less than or equal to 200 Daltons, such as from 100 to 200 Daltons; and/or
(b) a rejection rate for a material having a molecular weight that is at least 10% above the molecular weight cut-off value of the membrane is at least 90%.

23. The membrane according to claim 1, wherein the membrane is suitable for use in nanofiltration applications using one or more solvents selected from two or more of the solvent classes: polar protic, polar aprotic and non-polar organic solvents.

24. (canceled)

25. The membrane according to claim 1, wherein the membrane has a pure solvent permeance value of from 0.1 to 4 L m⁻² h⁻¹ bar⁻¹, optionally wherein, the membrane has one or more of the following pure solvent permeance values:
(a) from 2 - 4 L m⁻² h⁻¹ bar⁻¹ for methanol;
(b) from 0.5 - 1.5 L m⁻² h⁻¹ bar⁻¹ for isopropanol (IPA);
(c) from 0.6 - 4 L m⁻² h⁻¹ bar⁻¹ for hexane;
(d) from 0.9 - 1.5 L m⁻² h⁻¹ bar⁻¹ for toluene;
(e) from 0.8-1.3 L m⁻² h⁻¹ bar⁻¹ for tetrahydrofuran (THF);
(f) from 0.2-1 L m⁻² h⁻¹ bar⁻¹ for dimethylformamide (DMF); and
(g) from 1 - 2 L m ² h⁻¹ bar⁻¹ for ethanol.

26. A method of filtration using an organic solvent nanofiltration (OSN) membrane as described in claim 1, comprising the steps of
(a) providing a solution comprising a first compound having a first molecular weight and a second compound having a second molecular weight; and
(b) subjecting the solution to filtration using an OSN membrane as described in claim 1, such that the first and second compounds are separated from one another,
wherein the first molecular weight is lower than the second molecular weight and the OSN membrane has a molecular weight cut-off that prevents the second compound from passing through the membrane, thereby separating the first and second compounds.

27. (canceled)

28. (canceled)

29. A method of forming an organic solvent nanofiltration (OSN) membrane and/or an organic solvent reverse osmosis (OSRO) membrane comprising a mixed matrix material as described in claim 1, wherein the method comprises the steps of:
(a) providing a material comprising:
a partially crosslinked solvent-resistant polymeric matrix with functional groups suitable for crosslinking; and
an inorganic material homogeneously dispersed throughout the polymeric matrix, where the material has a first region comprising a surface of the material and a second region; and
(b) exposing the first region of the material to a vapour comprising a crosslinking agent that generates further crosslinks in said first region to provide the organic solvent nanofiltration (OSN) membrane.

30. A method of filtration using an organic solvent reverse osmosis (OSRO) membrane as described in claim 1, comprising the steps of
(a) providing a solution comprising a first solvent having a first molecular weight and a second solvent having a second molecular weight; and
(b) subjecting the solution to filtration using an OSRO membrane as described in claim 1, such that the first and second solvents are separated from one another,
wherein the first molecular weight is lower than the second molecular weight and the OSRO membrane has a molecular weight cut-off that prevents the second solvent from passing through the membrane, thereby separating the first and second solvents.

31. A method of forming an organic solvent reverse osmosis (OSRO) membrane comprising a mixed matrix material as described in claim 1, wherein the method comprises the steps of:
(a) providing a material comprising:
a partially crosslinked solvent-resistant polymeric matrix with functional groups suitable for crosslinking; and
an inorganic material homogeneously dispersed throughout the polymeric matrix, where the material has a first region comprising a surface of the material and a second region; and
(b) exposing the first region of the material to a vapour comprising a crosslinking agent that generates further crosslinks in said first region to provide the organic solvent reverse osmosis (OSRO) membrane.
